SEQUENCE LISTING (3 sequences)

SEQ ID NO: 1 - protein, 12 aa, Artificial sequence
  synthetic construct
GGGCYFQNCPKG

SEQ ID NO: 2 - protein, 13 aa, Artificial sequence
  synthetic construct
GGGCYFQNCPHKG

SEQ ID NO: 3 - protein, 9 aa, Artificial sequence
  synthetic construct
CYFQNCPRG

(19) United States
(12) Patent Application Publication
(10) Pub. No.: US 2023/0399360 A1
(43) Pub. Date: Dec. 14, 2023

(54) TERLIPRESSIN-OCTADECANEDIOIC ACID CONJUGATE FOR VASOCONSTRICTIVE THERAPY

(71) Applicant: Northwestern University, Evanston, IL (US)

(72) Inventors: Or BERGER, Evanston, IL (US); Wonmin CHOI, Evanston, IL (US); Nathan C. GIANNESCHI, Evanston, IL (US); Daniel BATLLE, Evanston, IL (US)

(73) Assignee: Northwestern University, Evanston, IL (US)

(21) Appl. No.: 17/785,518

(22) PCT Filed: Dec. 17, 2020
(86) PCT No.: PCT/US20/65692

§ 371 Date: Jun. 15, 2022

Related U.S. Application Data
(60) Provisional application No. 62/951,551, filed on Dec. 20, 2019.

(51) Int. Cl. C07K 7/16; A61K 47/54; A61K 38/38; A61P 9/00

(52) U.S. Cl. CPC C07K 7/16; A61K 47/542; A61K 38/385; A61P 9/00

(57) ABSTRACT

In an aspect, disclosed herein is a compound characterized by formula (FX1): A¹-X¹—X²-A² (FX1); wherein: A¹ is a carboxylic acid group, a carboxylate anion, or a carboxylate ester, X¹ is a substituted or unsubstituted and saturated or unsaturated C₁-C₅₀ aliphatic group; X² is a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin.

### CROSS-REFERENCE TO RELATED APPLICATIONS

This application is a U.S. National Stage Application under 35 U.S.C. § 371 of International Application No. PCT/US2020/065692, filed Dec. 17, 2020, which claims the benefit of priority to U.S. Provisional Patent Application No. 62/951,551, filed Dec. 20, 2019, each of which is hereby incorporated by reference in its entirety.

### STATEMENT REGARDING FEDERALLY SPONSORED RESEARCH OR DEVELOPMENT

None.

### BACKGROUND OF INVENTION

Terlipressin is a cyclic dodecamer peptide that interacts with multiple receptors in the body causing narrowing of blood vessels which leads to a rise in blood pressure. It also regulates reabsorption of water in the renal medulla, preventing excessive loss of water in the urine. U.S. Pat. No. 9,090,064, for example, describes terlipressin analogues and reports activity for several terlipressin amide and ester analogues. It is relevant to treating a variety of conditions, such as bleeding esophageal varices, septic shock, hepatorenal syndrome and management of low blood pressure. Terlipressin is a registered drug in Europe, Australia and parts of Asia, prescribed for patients with bleeding esophageal varices (bleeding from dilated veins in the food pipe leading to the stomach).

For example, if the vessels in the liver are blocked due to liver damage, blood cannot flow properly through the liver. As a result, high pressure in the portal system develops. This increased pressure in the portal vein may lead to the development of large, swollen veins (varices) within the esophagus, stomach, rectum, or umbilical area. Varices can rupture and bleed, resulting in potentially life-threatening complications. Bleeding of esophageal varices is one of the most dramatic complications in gastroenterology and has a 20-50% mortality rate, closely related to failure to control initial bleeding or early re-bleeding occurring in up to 30-40% of patients. The only approved drugs to arrest variceal bleeding are vasopressin and terlipressin. Treatment with terlipressin is preferable due to better efficacy, longer effects and less adverse effects compared to vasopressin. US 2019/0328831, for example, describes treatment of liver disease using terlipressin.

Treatment with terlipressin has several drawbacks, however. The potency of terlipressin is limited due to being readily digested in the human body because it is highly susceptible to serum and tissue proteases and is thus rapidly cleared from the circulation, typically in a matter of minutes. For example, the distribution half-life can be 8 minutes, while the elimination half-life can be 6 minutes. Consequently, terlipressin is typically administered by intermittent intravenous dosing schedule of approximately every 3-4 or 4-6 hours in doses of 1-2 mg per injection, until bleeding is under control. Duration of treatment can last up to 3 days. This frequency of drug administration results in significant cost and discomfort, and may require hospitalization.

There is thus need to improve treatments using terlipressin, or its analogues, by retaining the efficacy and tolerability of the drug and prolonging its potency, such that the number of administrations and overall treatment time are reduced.

### SUMMARY OF THE INVENTION

Included herein are compounds and pharmaceutical compositions therewith which address the challenges noted above, and others. For example, the compounds and pharmaceutical compositions included herein may have a longer half-life in a subject, thereby allowing for a reduced frequency of administration, compared to terlipressin. This, in turn, may make the procedure an out-patient procedure, save cost, and cause less discomfort to the subject compared to a conventional terlipressin drug.

Aspects of the invention include a compound characterized by formula (FX1): A¹-X¹—X²-A² (FX1); wherein: A¹ is a carboxylic acid group, a carboxylate anion, or a carboxylate ester; X¹ is a substituted or unsubstituted and saturated or unsaturated C₁-C₅₀ aliphatic group; X² is a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin. Also disclosed herein is a pharmaceutically acceptable salts of the compound. Optionally, X² is a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, and —N(═O)—. Optionally, in any of the embodiments herein, the peptide A² comprises a sequence having 50% or greater, 60% or greater, 70% or greater, 80% or greater, 90% or greater, or 95% or greater sequence homology of Seq. ID. 1 (GGGCYFQNCPKG). Preferably but not necessarily, the peptide A² comprises a sequence having 80% or greater sequence homology of Seq. ID. 1 (GGGCYFQNCPKG). Optionally, the peptide A² comprises the amino acid sequence of Seq. ID. 1 (GGGCYFQNCPKG).

Optionally, X² is selected from the group consisting of an amide group, an ester group, a disulfide group, a carbamate group, a carbonate group, a ketone group, and a combination thereof. Optionally, X² is a ketone group (—C(═O)—).

Optionally, X¹ is a substituted or unsubstituted and saturated or unsaturated C₁₀-C₃₀ aliphatic group. Optionally, X¹ is a substituted or unsubstituted C₁₀-C₃₀ alkylene group. Optionally, X¹ is fully saturated. Optionally, X¹ comprises a number of pi bonds selected from the range of 1 to 3 or wherein X¹ is characterized by a degree of unsaturation selected from the range of 1 to 3. Optionally, X¹ is unsubstituted. Optionally, X¹ comprises a number of substituents selected from the range of 1 to 3. Optionally, X¹ is (CH₂)₁₂, (CH₂)₁₄, (CH₂)₁₆, (CH₂)₁₈, (CH₂)₂₀, or (CH₂)₂₂. Optionally, X¹ is (CH₂)₁₆.

Optionally, A² is terlipressin, vasopressin, omipressin, desmopressin, lypressin, or felypressin. Optionally, the peptide A² is a substituted or unsubstituted derivative, a substituted or unsubstituted natural or synthetic analogue, a substituted or unsubstituted variant, a substituted or unsubstituted isomer, or a substituted or unsubstituted fragment of terlipressin. Optionally, the peptide A² comprises a sequence having 50% or greater, 60% or greater, 70% or greater, 80% or greater, 90% or greater, or 95% or greater sequence homology of SEQ. ID. NO: 1 (GGGCYFQNCPKG). Preferably but not necessarily, the peptide A² comprises a sequence having 80% or greater sequence homology of SEQ. ID. NO: 1 (GGGCYFQNCPKG). Optionally, the peptide A² comprises the amino acid sequence of SEQ. ID. NO: 1 (GGGCYFQNCPKG). Optionally, X² is covalently bound to an amine group of A². Optionally, X² is covalently bound to an N-terminus of A². Optionally, X² is covalently bound to a C-terminus of A².

Optionally, the compound is characterized by the formula (FX2):

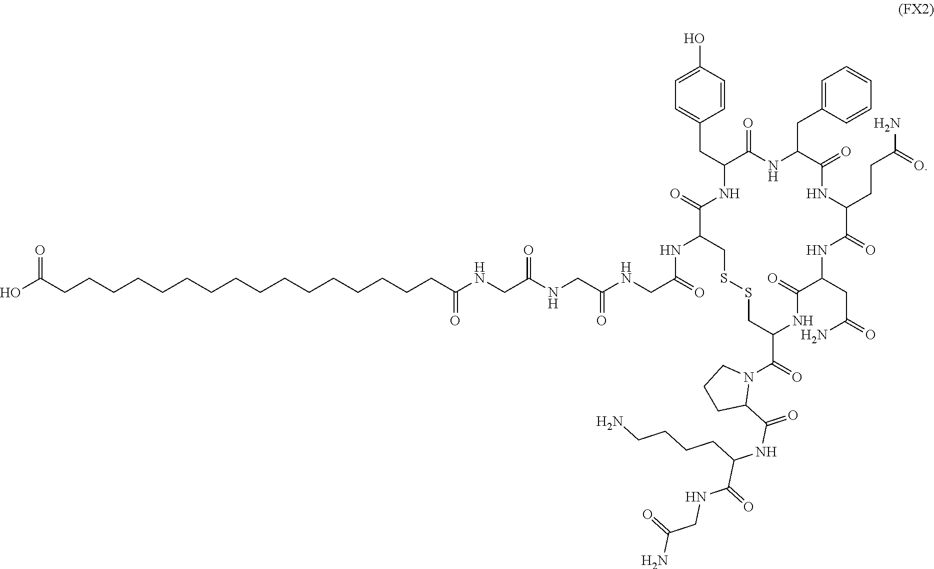 [SMILES: NCCCCC(NC(=O)C1CCCN1C(=O)C1CSSCC(NC(=O)CNC(=O)CNC(=O)CNC(=O)CCCCCCCCCCCCCCCCC(=O)O)C(=O)NC(Cc2ccc(O)cc2)C(=O)NC(Cc2ccccc2)C(=O)NC(CCC(N)=O)C(=O)NC(CC(N)=O)C(=O)N1)C(=O)NCC(N)=O]

Aspects of the invention include a pharmaceutical composition comprising: a compound characterized by formula (FX1): A¹-X¹—X²-A² (FX1); wherein: A¹ is a carboxylic acid group, a carboxylate anion, or a carboxylate ester; X¹ is a substituted or unsubstituted and saturated or unsaturated C₁-C₅₀ aliphatic group; X² is a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin. Optionally, X² is a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, and —N(═O)—. Optionally, any of the pharmaceutical compositions further comprise a protein, wherein the protein is human serum albumin or a protein whose sequence is at least 50% equivalent to that of human serum albumin. Optionally, the protein is human serum albumin (HSA) or a human serum albumin mimetic. Optionally, the protein is human serum albumin (HSA). Optionally, the protein has a sequence that is at least 50% equivalent to that of HSA, or at least 60% equivalent to that of HSA, or at least 70% equivalent to that of HSA, or at least 80% equivalent to that of HSA, or at least 90% equivalent to that of HSA, or at least 95% equivalent to that of HSA, at least 97% equivalent to that of HSA, at least 99% equivalent to that of HSA. Optionally, the compound and the protein are non-covalently associated with each other. Optionally, the A¹ of the compound is non-covalently associated with the protein. Optionally, pharmaceutical composition comprises a carrier, such as a liquid carrier. Optionally, the carrier comprises water. For example, in some such embodiments, water makes up at least 50% by volume, or at least 60% by volume, or at least 70% by volume, or at least 80% by volume, or at least 90% by volume, based on the total volume of liquid materials in the pharmaceutical composition. The carrier can also include other liquid ingredients, such as liquid ingredients commonly included in aqueous pharmaceutical formulations for parenteral administration. Optionally, the compound and the protein are solvated by the carrier. Preferably, the pharmaceutical composition is suitable for intravenous administration to a mammal. Optionally, the pharmaceutical composition is capable of stimulating a V1 receptor in a vascular smooth muscle cell. For example, the pharmaceutical composition is preferably a V1 receptor agonist. For example, the compound is preferably a V1 receptor agonist). Terlipressin is a stimulator for smooth muscle cells. HSA can be administered in parallel with the compound to HRS patients because doing so can improve the kidney function. The compound can also be administered without a protein.

In some embodiments, the compound of formula (FX1) and the protein (e.g., human serum albumin) are non-covalently associated with each other with a binding constant (Kb) of at least 10² M⁻¹, or at least 10³ M⁻¹, or at least 10⁴ M⁻¹, or at least 10⁵ M⁻¹ at 25° C. in the aqueous composition.

In some embodiments having an aqueous carrier, the compound of formula (FX1) and the protein are solvated by the carrier. In some such embodiments, at least 90% by weight, or at least 95% by weight, or at least 97% by weight, or at least 98% by weight, or at least 99% by weight of the compounds of formula (FX1) in the composition are bound non-covalently to the protein with a binding constant (Kb) of at least 10² M″¹, or at least 10³ M″¹, or at least 10⁴ M″¹, or at least 10⁵ M″¹ at 25° C. in the aqueous composition.

The compound of formula (FX1) can have any suitable molar ratio to the protein in the pharmaceutical composition. For example, in some embodiments, the molar ratio of the compound of formula (FX1) to the protein ranges from 1:10 to 20:1, or from 1:5 to 15:1, or from 1:2 to 10:1. In some embodiments, the molar ratio of the compound of formula (FX1) to the protein is about 1:1, or is about 2:1, or is about 3:1, or is about 4:1, or is about 5:1, or is about 6:1, or is about 7:1, wherein the term “about,” in this instance means±0.5:1, such that “about 5:1” refers to a range from 4.5:1 to 5.5:1.

Optionally, the pharmaceutical composition has a half-life that is at least 5% longer than the half-life of terlipressin in mammal blood after an administration using otherwise identical conditions. Optionally, the pharmaceutical composition has a half-life that is at least 10% longer, preferably at least 20% longer, more preferably at least 30% longer, still more preferably at least 50% longer, further more preferably at least 100% longer than the half-life of terlipressin in mammal blood after an administration using otherwise identical conditions (e.g., including identical concentrations and administration method, etc.). Optionally, the pharmaceutical composition is capable of having a half-life that is greater than 1 hour in living mammal blood. Optionally, the pharmaceutical composition is capable of causing an increased systolic blood pressure in mammals for at least 5 minutes, preferably at least 10 minutes, more preferably at least 20 minutes, still more preferably at least 30 minutes, and further more preferably at least 60 minutes longer than terlipressin after an administration using otherwise identical conditions. Optionally, the pharmaceutical composition is capable of causing an increased systolic blood pressure in mammals for at least 5 minutes longer than terlipressin after an administration using otherwise identical conditions.

In certain aspects, the compounds of any of the embodiments may be formulated into pharmaceutical compositions in any suitable manner. In general, but not necessarily, such pharmaceutical formulations are aqueous formulations suitable for parenteral administration, such as intravenous or intra-arterial administration.

In another aspect, also disclosed herein is a method of treating or managing a condition in a living subject, the method comprising steps of: administering to the subject a pharmaceutical composition; wherein the pharmaceutical composition comprises: a compound characterized by formula (FX1): A¹-X¹—X²-A² (FX1); wherein: A¹ is a carboxylic acid group, a carboxylate anion, or a carboxylate ester; X¹ is a substituted or unsubstituted and saturated or unsaturated C₁-C₅₀ aliphatic group; X² is a linker group selected from the group consisting of a direct bond, an organic group, —O—, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin. Optionally, X² is a linker group selected from the group consisting of a direct bond, an organic group, —O—, —S—, —S(═O)—, —S(═O)₂—, —S—S—, —N═, ═N—, —N(H)—, —N═N—N(H)—, —N(H)—N═N—, —N(OH)—, and —N(═O)—. The condition is optionally selected from the group consisting of hepatorenal syndrome, low blood pressure, bleeding esophageal varices, septic shock paracentesis-induced circulatory dysfunction, a condition or disease that can be treated using vasoconstriction or albumin-mediated vasoconstriction, and any combination thereof. The condition is optionally selected from the group consisting of hepatorenal syndrome, central diabetes insipidus, low blood pressure, bleeding esophageal varices, septic shock paracentesis-induced circulatory dysfunction, a condition or disease that can be treated using vasoconstriction or albumin-mediated vasoconstriction, and any combination thereof. Optionally, the living subject is a mammal. Optionally, the step of administering comprises administrating a pharmaceutically effecting amount of the pharmaceutical composition to the living subject. Optionally, the step of administering comprises intravenous administration in the living subject. Optionally, the step of administering is performed at a frequency of greater than 6 hours. Optionally, the pharmaceutical composition stimulates a V1 receptor in a vascular smooth muscle cell. Optionally, the pharmaceutical composition has a half-life that is greater than 1 hour in the blood of the subject after being administered. Optionally, the pharmaceutical composition causes an increased systolic blood pressure in the subject for at least 20 minutes, preferably at least 30 minutes, more preferably at least 1 hour, still more preferably at least 6 hours, more preferably at least 12 hours, and further more preferably at least 24 hours after being administered. Optionally, the pharmaceutical composition causes an increased systolic blood pressure in the subject for at least 30 minutes after being administered. Optionally, the pharmaceutical composition further comprises a protein, wherein the protein is human serum albumin or a protein whose sequence is at least 50% equivalent to that of human serum albumin; wherein the compound and the protein are non-covalently associated with each other. Optionally, the pharmaceutical composition further comprises a carrier; wherein the carrier comprises water.

In some aspects, the disclosure provides uses of a compound or composition of any of the aspects and embodiments disclosed herein as a medicament. In some aspects, the disclosure provides uses of a compound or composition of any of the aspects and embodiments disclosed herein in the manufacture of a medicament.

Aspects of the invention include a method for making a compound, the method comprising: conjugating a molecule comprising A¹-X¹—X²— with A², thereby forming the compound; wherein the compound is characterized by formula (FX1): A¹-X¹—X²-A² (FX1); wherein: A¹ is a carboxylic acid group, a carboxylate anion, or a carboxylate ester; X¹ is a substituted or unsubstituted and saturated or unsaturated C₁-C₅₀ aliphatic group; X² is a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin. Optionally, X² is a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, and —N(═O)—. Optionally, the molecule is conjugated to amine group of A². Optionally, the molecule is conjugated to an N-terminus of A². Optionally, X² is covalently bound to a C-terminus of A². Optionally, the molecule is mono-protected during the step of conjugating. Optionally, the molecule is unprotected during the step of conjugating. Optionally, the molecule is in a cyclic anhydride form during the step of conjugating.

Aspects of the invention include a compound characterized by formula (FX10): (A¹-X¹—X²—)ₙA² (FX10); wherein: each A¹ is independently a carboxylic acid group, a carboxylate anion, or a carboxylate ester; each X¹ is independently a substituted or unsubstituted and saturated or unsaturated C₁-C₅₀ aliphatic group; each X² is independently a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin; and n is an integer selected from the range of 1 to 10. Optionally, n is an integer selected from the range of 1 to 5. Optionally, in is an integer selected from the range of 2 to 10. Optionally, X² is a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, and —N(═O)—. Optionally, n is greater than 1 and each (A¹-X¹—X²—) is independently covalently bound to a unique binding site, or point of attachment, of A². Optionally, each (A¹-X¹—X²—) is independently covalently bound to a unique amine group of A². The composition or formula of each (A¹-X¹—X²—) is independently according to any of the respective embodiments corresponding to (A¹-X¹—X²—) disclosed herein (e.g., such as any of those shown in Table 2). A binding site corresponds to a location, site, or attachment point at the peptide A² at which or to which (A¹-X¹—X²—) can be chemically bound. In other words, optionally, the compound of formula FX10 can have the A² peptide moiety conjugated with more than one (A¹-X¹—X²—) moiety, wherein each of the more than one (A¹-X¹—X²—) moiety is independently unique or is identical to another (A¹-X¹—X²—) moiety. Preferably, each (A¹-X¹—X²—) moiety is bound (preferably covalently bound) to the A² peptide moiety at a different attachment point than any other (A¹-X¹—X²—) moiety on the same A² peptide moiety in the compound having formula FX10. Each (A¹-X¹—X²—) moiety can be bound or attached to the A² peptide moiety at one of a variety of functional groups, wherein the result is not sterically impractical and/or synthetically non-feasible. Each (A¹-X¹—X²—) moiety of the compound of formula FX10 can be according to any one or any combination of embodiments of a (A¹-X¹—X²—) moiety described herein.

Aspects of the invention include a pharmaceutical composition comprising: a compound characterized by formula (FX10): (A¹-X¹—X²-)ₙA² (FX10); wherein: each A¹ is independently a carboxylic acid group, a carboxylate anion, or a carboxylate ester; each X¹ is independently a substituted or unsubstituted and saturated or unsaturated C₁-C₅₀ aliphatic group; each X² is independently a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin; and n is an integer selected from the range of 1 to 10. Optionally, n is an integer selected from the range of 1 to 5. Optionally, in is an integer selected from the range of 2 to 10. Optionally, X² is a linker group selected from the group consisting of a direct bond, an organic group, -0-, —S—, —S(═O)—, —S(═O)₂—, —S—S—, —N═, ═N—, —N(H)—, —N═N—N(H)—, —N(H)—N═N—, —N(OH)—, and —N(═O)—. Optionally, n is greater than 1 and each (A¹-X¹—X²—) is independently covalently bound to a unique binding site, or point of attachment, of A². Optionally, each (A¹-X¹—X²—) is independently covalently bound to a unique amine group of A². Optionally, the pharmaceutical composition further comprises a protein, wherein the protein is human serum albumin or a protein whose sequence is at least 50% equivalent to that of human serum albumin. The composition or formula of each (A¹-X¹—X²—) is independently according to any of the respective embodiments corresponding to (A¹-X¹—X²—) disclosed herein (e.g., such as any of those shown in Table 2).

Also disclosed herein are compounds according to any one or any combination of embodiments of compounds disclosed herein. Also disclosed herein are pharmaceutical compositions according to any one or any combination of embodiments of compounds, pharmaceutical compositions, or combinations thereof, disclosed herein. Also disclosed herein are methods including any one or any combination of embodiments of compounds, pharmaceutical compositions, methods, or combinations thereof, disclosed herein.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A. Chemical structure of octadecanedioic acid-terlipressin conjugate. FIG. 1B. The conjugate is proposed to bind to HAS at several binding sites for long chain fatty acids. FIG. 1C. Calcium influx assay in human aortic smooth muscle cells as a response to the addition of terlipressin compared to a conjugated terlipressin, an exemplary compound according to certain embodiments disclosed herein. In FIG. 1C, the label “C18-conjugate” and “ODDA-Terlipressin conjugate” both refer to octadecanedioic acid-terlipressin, or terlipressin conjugated with octadecanedioic acid, according to embodiments disclosed herein.

FIGS. 2A-2B. Systolic blood pressure in mice was measured following injections of (FIG. 2A) octadecanedioic acid-terlipressin conjugate compared to PBS; (FIG. 2B) octadecanedioic acid-terlipressin compared to terlipressin peptide alone.

FIG. 3. Schematic of octadecanedioic acid-terlipressin conjugate and an illustration of the same bound to an albumin protein.

FIG. 4. Schematic representing a method for making and characterizing a compound according to certain embodiments, where terlipressin (represented by SEQ. ID. NO: 2) is conjugated with a fatty acid.

FIG. 5. Illustration of an interaction of terlipressin with a V1 receptor.

FIGS. 6A-6B. Calcium flux assay in human aortic smooth muscle cells. FIG. 6A: comparing terlipressin and a conjugated terlipressin, an exemplary compound according to certain embodiments disclosed herein. FIG. 6B: comparing different concentrations of a conjugated terlipressin, according to certain embodiments disclosed herein.

FIGS. 7A-7B. Systolic blood pressure vs. time comparing PBS and a conjugated terlipressin, an exemplary compound according to certain embodiments disclosed herein (FIG. 7A). Systolic blood pressure vs. time comparing PBS and terlipressin.

FIG. 8. Systolic blood pressure vs time data comparing efficacy of terlipressin with a conjugated terlipressin, an exemplary compound according to certain embodiments disclosed herein.

FIG. 9. An illustration of a sample preparation procedure, according to certain embodiments, for pharmacokinetics testing.

FIGS. 10A-10B. Liquid chromatography with tandem mass spectrometry data (intensity vs. time) collected at 1 hour after administration, comparing signal corresponding to terlipressin (FIG. 10A) and a conjugated terlipressin (FIG. 10B), an exemplary compound according to certain embodiments disclosed herein. The label “C18-conjugate” refers to octadecanedioic acid-terlipressin, or terlipressin conjugated with octadecanedioic acid, according to embodiments disclosed herein.

FIGS. 11A-11C. Timeline (FIG. 11A), table (FIG. 11B), and plot (FIG. 11C) of concentration vs. time corresponding to pharmacokinetic measurements of concentration of a conjugated terlipressin, an exemplary compound according to certain embodiments disclosed herein, in plasma of animals showing that the half-lifetime of the conjugated terlipressin in the plasma is about 1 hour. The label “C18-conjugate” refers to octadecanedioic acid-terlipressin, or terlipressin conjugated with octadecanedioic acid, according to embodiments disclosed herein.

FIG. 12. Formulas corresponding to vasopressin (SEQ ID NO:3) and to terlipressin (SEQ ID NO: 1).

FIG. 13. An illustration of vascular smooth muscle cell components, adapted from Ertmer et al. Yearbook of intensive care and emergency medicine (2008).

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

The term “sequence homology” or “sequence identity” means the proportion of amino acid matches between two amino acid sequences. When sequence homology is expressed as a percentage, e.g., 50%, the percentage denotes the fraction of matches over the length of sequence that is compared to some other sequence. Gaps (in either of the two sequences) are permitted to maximize matching; for example, wherein gap lengths of 5 amino acids or less, optionally 3 amino acids or less, are usually used.

The term “fragment” refers to a portion, but not all of, a composition or material, such as a peptide composition or material. In an embodiment, a fragment of a polypeptide refers to 50% or more of the sequence of amino acids, optionally 70% or more of the sequence of amino acids and optionally 90% or more of the sequence of amino acids. In embodiments wherein the therapeutic peptide is a fragment of a vasopressin analogue, the fragment can be any suitable fragment. Optionally, the fragment can be any suitable fragment, for example 2-8 amino acid units, for example, 8 or less, 7 or less, 6 or less, 5 or less, 4 or less, 3 or less, or 2 amino acid units.

The term “analogue” refers to a composition, compound, or molecule, such as a peptide, that is structurally or chemically similar to base composition, compound, or molecule, such as a base peptide. The analogue can be a natural analogue. The analogue can be a synthetic analogue. In embodiments, the analogue has five or fewer substituted or unsubstituted amino acids, or derivatives thereof, that are different, removed, added, or any combination of these, with respect to the base. For example, an analogue of terlipressin can have five or fewer substituted or unsubstituted amino acids, or derivatives thereof, that are different, removed, added, or any combination of these, with respect to terlipressin.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

An “effective amount” is an amount sufficient to accomplish a stated purpose (e.g. achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g. transcription factor) relative to the absence of the antagonist. An “activity increasing amount,” as used herein, refers to an amount of agonist (activator) required to increase the activity of an enzyme or protein (e.g. transcription factor) relative to the absence of the agonist. A “function disrupting amount,” as used herein, refers to the amount of antagonist (inhibitor) required to disrupt the function of an enzyme or protein (e.g. transcription factor) relative to the absence of the antagonist. A “function increasing amount,” as used herein, refers to the amount of agonist (activator) required to increase the function of an enzyme or protein (e.g. transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As defined herein, the term “inhibition”, “inhibit”, “inhibiting” and the like in reference to a protein-inhibitor (e.g. antagonist) interaction means negatively affecting (e.g. decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein

The term “modulator” refers to a composition that increases or decreases the level of a target molecule or the function of a target molecule.

“Patient”, “subject, or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, cats, monkeys, other primates, goat, sheep, cows, deer, and other non-mammalian animals. In some embodiments, a subject is human. In some embodiments, a patient is a mammal. In some embodiments, a patient is a mouse. In some embodiments, a patient is an experimental animal. In some embodiments, a patient is a rat. In some embodiments, a patient is a test animal. The term “subject” does not require one to have any particular status with respect to a hospital, clinic, or research facility (e.g., as an admitted patient, a study participant, or the like).

As used herein, the term “pharmaceutical composition” is used to denote a composition that may be administered to a subject, such as a mammalian host, such as orally, topically, parenterally, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal, subcutaneous administration, intracisternal delivery, delivery by infusion techniques, transdermal delivery, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent or chemotherapeutic). The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). Depending on the mode of delivery, the administering can be carried out by various individuals, including, for example, a health-care professional (e.g., physician, nurse, etc.), a pharmacist, or the subject (i.e., self-administration).

As used herein, the term “conjugated” when referring to two chemical species or moieties means the two chemical species or moieties are bonded, wherein the bond or bonds connecting the two chemical species or moieties may be covalent or non-covalent. In embodiments, the two chemical species or moieties are covalently bonded to each other (e.g. directly or through a covalently bonded intermediary). In embodiments, the two chemical species or moieties are non-covalently bonded (e.g. through ionic bond(s), Van der Waal's bond(s) interactions, hydrogen bond(s), polar bond(s), or combinations or mixtures thereof). The term “conjugating” refers to the causing of two chemical species or moieties to become conjugated.

Unless otherwise specified, the term “average molecular weight,” refers to number average molecular weight. Number average molecular weight is the defined as the total weight of a sample volume divided by the number of molecules within the sample. As is customary and well known in the art, peak average molecular weight and weight average molecular weight may also be used to characterize the molecular weight of the distribution of polymers within a sample.

The term “degree of unsaturation” refers to a calculation corresponding to a measure of unsaturation of a compound, moiety, or group, representing a total number rings and pi (Tr) bonds in the compound, moiety, or group. The degree of unsaturation (DU) can be determined using the formula: DU=1+½Σnᵢ(υᵢ−2), where nᵢ is the number of atoms with valence υᵢ.

The term “wt. %” or “wt %” refers to a weight percent, or a mass fraction represented as a percentage by mass. The term “at. %” or “at %” refers to an atomic percent, or an atomic ratio represented as a percentage of a type of atom with respect to total atoms in a given matter, such as a molecule, compound, material, nanoparticle, polymer, dispersion, etc.

As used herein, the term “polymer” refers to a molecule composed of repeating structural units connected by covalent chemical bonds often characterized by a number of repeating units, also referred to as base units (e.g., greater than or equal to 2 base units). As used herein, a term “polymer” is inclusive of an “oligomer” (i.e., an oligomer is a polymer; i.e., a polymer is optionally an oligomer). An “oligomer” refers to a molecule composed of repeating structural units, also referred to as base units, connected by covalent chemical bonds often characterized by a number of repeating units less such that the oligomer is a low molecular weight polymer. Preferably, but not necessarily, for example, an oligomer has equal to or less than 100 repeating units. Preferably, but not necessarily, for example, an oligomer has a lower molecular weight less than or equal to 10,000 Da. Oligomers may be the polymerization product of one or more monomer precursors. Polymerization of one or more monomers, or monomer precursors, resulting in formation of an oligomer may be referred to as oligomerization. An oligomer optionally includes 100 or less, 50 or less, 15 or less, 12 or less, 10 or less, or 5 or less repeating units (or, “base units”). An oligomer may be characterized has having a molecular weight of 10,000 Da or less, 5,000 Da or less, 1,000 Da or less, 500 Da or less, or 200 Da or less. A dimer, a trimer, a tetramer, or a pentamer is an oligomer having two, three, four, or five, respectively, repeating units, or base units. Polymers can have, for example, greater than 100 repeating units. Polymers can have, for example, a high molecular weight, such as greater than 10,000 Da, in some embodiments greater than or equal to 50,000 Da or greater than or equal to 100,000 Da. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits, and include random, block, brush, brush block, alternating, segmented, grafted, tapered and other architectures. Useful polymers include organic polymers or inorganic polymers that may be in amorphous, semi-amorphous, crystalline or semi-crystalline states. Polymer side chains capable of cross linking polymers (e.g., physical cross linking) may be useful for some applications. The terms “monomer unit,” “repeating monomer unit,” “repeating unit,” and “polymerized monomer” can be used interchangeably and refer to a monomeric portion of a polymer described herein which is derived from or is a product of polymerization of one individual “monomer” or “polymerizable monomer.” Each individual monomer unit of a polymer is derived from or is a product of polymerization of one polymerizable monomer. Each individual “monomer unit” or “repeating unit” of a polymer comprises one (polymerized) polymer backbone group. For example, in a polymer that comprises monomer units X and Y arranged as X-Y-X-Y-X-Y-X-Y (where each X is identical to each other X and each Y is identical to each other Y), each X and each Y is independently can be referred to as a repeating unit or monomer unit.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

The term “moiety” refers to a group, such as a functional group, of a chemical compound or molecule. A moiety is a collection of atoms that are part of the chemical compound or molecule. The present invention includes moieties characterized as monovalent, divalent, trivalent, etc. valence states. Generally, but not necessarily, a moiety comprises more than one functional group.

As used herein, the term “substituted” refers to a compound wherein one or more hydrogens is replaced by another functional group, provided that the designated atom's normal valence is not exceeded. An exemplary substituent includes, but is not limited to: a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. In some embodiments, the term substituted refers to a compound wherein each of more than one hydrogen is replaced by another functional group, such as a halogen group. For example, when the substituent is oxo (i.e., ═O), then two hydrogens on the atom are replaced. The substituent group can be any substituent group described herein. For example, substituent groups can include one or more of a hydroxyl, an amino (e.g., primary, secondary, or tertiary), an aldehyde, a carboxylic acid, an ester, an amide, a ketone, nitro, an urea, a guanidine, cyano, fluoroalkyl (e.g., trifluoromethane), halo (e.g., fluoro), aryl (e.g., phenyl), heterocyclyl or heterocyclic group (i.e., cyclic group, e.g., aromatic (e.g., heteroaryl) or non-aromatic where the cyclic group has one or more heteroatoms), oxo, or combinations thereof. Combinations of substituents and/or variables are permissible provided that the substitutions do not significantly adversely affect synthesis or use of the compound.

As used herein, the term “derivative” refers to a compound wherein an atom or functional group is substituted or replaced by another atom or functional group (e.g., a substituent function group as also described below), including, but not limited to: a hydrogen, a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. Optionally, the term “derivative” refers to a compound wherein one or two atoms or functional groups are independently replaced by another atom or functional group. Optionally, the term derivative does not refer to or include replacement of a chalcogen atom (S, Se) that is a member of a heterocyclic group. Optionally, the term derivative does not refer to or include replacement of a chalcogen atom (S, Se) nor a N (nitrogen) where the chalcogen atom and the N are members same heterocyclic group. Optionally, the term derivative does not include breaking a ring structure, replacement of a ring member, or removal of a ring member.

As is customary and well known in the art, hydrogen atoms in formulas, such as formula (FX2), are not always explicitly shown, for example, hydrogen atoms bonded to the carbon atoms of aromatic, heteroaromatic, and alicyclic rings are not always explicitly shown. The structures provided herein, for example in the context of the description of formula (FX2) and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, “hydrocarbon” refers to an organic group composed of carbon and hydrogen, which can be saturated or unsaturated, and can include aromatic groups. The term “hydrocarbyl” refers to a monovalent or polyvalent (e.g., divalent or higher) hydrocarbon moiety. In some cases, a divalent hydrocarbyl group is referred to as a “hydrocarbylene” group.

The number of carbon atoms in any group or compound can be represented by the terms. Thus, “C_{z}” refers to a group of compound having z carbon atoms, and “C_{x-y}”, refers to a group or compound containing from x to y, inclusive, carbon atoms. For example, “C₁₋₆ alkyl” represents an alkyl group having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. The same logic applies to other types of functional groups, defined below.

As used herein, the terms “organic group,” “organic moiety,” or “organic residue” refer to a monovalent or polyvalent functional group having at least one carbon atom. Optionally, the organic group contains one or more additional atoms such as, but not limited to, hydrogen atoms, halogen atoms, nitrogen atoms, oxygen atoms, phosphorus atoms, and sulfur atoms. Optionally, the organic group contains one or more additional atoms selected from the group consisting of hydrogen atoms, halogen atoms, nitrogen atoms, oxygen atoms, phosphorus atoms, and sulfur atoms, and which does not include covalently bound metal or semi-metal atoms. In some embodiments, these terms can include metal salts of organic groups, such as alkali metal or alkaline earth metal salts of organic anions.

As used herein, the term “pharmacophore” refers to a type of organic functional group. Standard pharmacophores are hydrophobic pharmacophores, hydrogen-bond donating pharmacophores, hydrogen-bond accepting pharmacophores, positive ionizable pharmacophores, and negative ionizable pharmacophores. The classification of organic functional groups within a compound is carried out according to standard classification systems known in the art.

As used herein, the terms “hydrophobic group,” “hydrophobic moiety,” or “hydrophobic residue” refer to an organic group that consists essentially of hydrophobic pharmacophores. In some embodiments, the terms refer to an organic group that consists of hydrophobic pharmacophores.

As used herein, the terms “hydrophilic group,” “hydrophilic moiety,” or “hydrophilic residue” refer to an organic group that comprises one pharmacophores selected from the group consisting of hydrogen bond donors, hydrogen bond acceptors, negative ionizable groups, or positive ionizable groups. In some embodiments, the terms refer to an organic group that consist essentially of pharmacophores selected from the group consisting of hydrogen bond donors, hydrogen bond acceptors, negative ionizable groups, or positive ionizable groups.

As used herein, the term “peptide moiety” refers to a peptide compound, or a pharmaceutically acceptable salt thereof, where an atom or a group of atoms is absent, thereby creating a monovalent or polyvalent moiety. As used herein, the term “peptide compound” refers to a compound formed from the condensation of two or more amino acid compounds. Any suitable amino acid compounds can be used, including L-amino acids or D-amino acids. In addition, the amino acids can be alpha-amino acids, beta-amino acids, gamma-amino acids, and delta-amino acids. In some embodiments, the amino acids are L-alpha-amino acids. In some embodiments, at least 80% by number, or at least 85% by number, or at least 90% by number, or at least 95% by number of the amino acids forming the peptide moiety are selected from the 22 proteinogenic amino acids. Conjugates can be formed in any suitable way. In some embodiments, for example, a hydrogen atom is absent from the N-terminal end of the peptide compound, thereby creating a monovalent moiety. A non-limiting example of such a “peptide moiety,” is the moiety of the following formula:

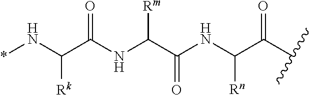

where a hydrogen atom is absent to create a monovalent moiety that, within a compound, bonds to the rest of the molecule through the remaining nitrogen atom, and wherein R^{k}, R^{m}, and Rⁿ are amino acid substituents. Note that the term “peptide moiety” is not limited to any particular procedure for making such compounds or moieties.

Various methods of drawing chemical structures are used herein. In some instances, the bond line-structure method is used to depict chemical compounds or moieties. In the line-structure method, the lines represent chemical bonds, and the carbon atoms are not explicitly shown (but are implied by the intersection of the lines). The hydrogen atoms are also not explicitly shown, except in instances where they are attached to heteroatoms. Heteroatoms, however, are explicitly shown. Thus, using that methodology, the structures shown below are for 2-methylpropane, 1-methoxypropane, and 1-propanol:

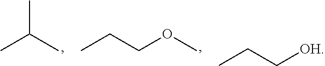

In that methodology, aromatic rings are typically represented merely by one of the contributing resonance structures. Thus, the following structures are for benzene, pyridine, and pyrrole:

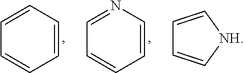

As used herein, a “protein binding moiety” is a moiety that binds non-covalently to one or more sites on a protein with a binding constant (Kb) of at least 100 M⁻¹ in water at 25° C.

As used herein, “amino acid” refers to a compound having the structure H₂N—R^{x}—COOH, where R^{x} is an organic group, and where the NH₂ may optionally combine with Rx (e.g., as in the case of proline). The term includes any known amino acids, including, but not limited to, alpha amino acids, beta amino acids, gamma amino acids, delta amino acids, and the like. In some embodiments, the term can refer to alpha amino acids. Amino acids include glycine, alanine, valine, leucine, isoleucine, methionine, proline, phenylalanine, tryptophan, asparagine, glutamine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides are comprised of two or more amino acids connected via peptide bonds. Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides and peptide moieties, as used and described herein, comprise two or more amino acid groups connected via peptide bonds.

Amino acids and amino acid groups refer to naturally-occurring amino acids, unnatural (non-naturally occurring) amino acids, and/or combinations of these. Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (lie), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof. Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to herein by either the commonly known three letter symbols or by the one-letter symbols recommended by the IUPAC-IUB Biochemical Nomenclature Commission.

As used herein, “hydroxy acid” refers to a compound having the structure HO—R^{y}—COOH, where R^{y} is an organic group. Non-limiting examples include glycolic acid, lactic acid, and caprolactone.

As used herein, “alkanol amine” refers to a compound having the structure HO—R^{z}—NH₂, where R^{z} is an optionally substituted alkylene group. Non-limiting examples include ethanol amine.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g. L¹-L²).

The term “aliphatic group” refers to a non-aromatic hydrocarbon group. An aliphatic group can be saturated or unsaturated. An aliphatic group can be cyclic or non-cyclic. Alkyl groups and alkylene groups, for example, are aliphatic groups.

As used herein, the terms “cylcoalkenylene” and “cylcoalkenylene group” are used synonymously and refer to a divalent group derived from a cylcoalkenyl group as defined herein. The invention includes compounds having one or more cylcoalkenylene groups. Cycloalkenylene groups in some compounds function as linking and/or spacer groups. Compounds of the invention include substituted and/or unsubstituted C₃-C₂₀ cylcoalkenylene, C₃-C₁₀ cylcoalkenylene and C₃-C₅ cylcoalkenylene groups, for example, as one or more linking groups (e.g. L¹-L⁶).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g. L¹-L²).

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g. L¹-L²).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g. L¹-L²).

As used herein, the terms “cycloalkenylene” and “cycloalkenylene group” are used synonymously and refer to a divalent group derived from a cycloalkenyl group as defined herein. The invention includes compounds having one or more cycloalkenylene groups. Cycloalkenylene groups in some compounds function as linking and/or spacer groups. Compounds of the invention include substituted and/or unsubstituted C₃-C₂₀ cycloalkenylene, C₃-C₁₀ cycloalkenylene and C₃-C₅ cycloalkenylene groups, for example, as one or more linking groups (e.g. L¹-L²).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g. L¹-L²).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)ₙ-alkoxy wherein n is an integer from 1 to 10, preferably 1 to 4, and more preferably for some embodiments 1 to 3.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—. Compositions of some embodiments of the invention comprise alkyl groups as terminating groups, such as polymer backbone terminating groups and/or polymer side chain terminating groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others:
halogen, including fluorine, chlorine, bromine or iodine;
pseudohalides, including —CN;
—COOR where R is a hydrogen or an alkyl group or an aryl group and more specifically where R is a methyl, ethyl, propyl, butyl, or phenyl group all of which groups are optionally substituted;
—COR where R is a hydrogen or an alkyl group or an aryl group and more specifically where R is a methyl, ethyl, propyl, butyl, or phenyl group all of which groups are optionally substituted;
—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;
—SR, where R is hydrogen or an alkyl group or an aryl group and more specifically where R is hydrogen, methyl, ethyl, propyl, butyl, or a phenyl group, which are optionally substituted;
—SO₂R, or —SOR where R is an alkyl group or an aryl group and more specifically where R is a methyl, ethyl, propyl, butyl, or phenyl group, all of which are optionally substituted;
—OCOOR where R is an alkyl group or an aryl group;
—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms; and
—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

Certain compounds, molecules, or groups disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or D- or L-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. Isomers include structural isomers and stereoisomers such as enantiomers.

The term “tautomer,” as used herein, refers to one of two or more structural isomers which exist in equilibrium and which are readily converted from one isomeric form to another. It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of one or more chemical moieties, one or more functional groups, one or more atoms, one or more ions, an unpaired electron, or one or more other chemical species to the re resented molecule, compound, or chemical formula. For example, in the formula

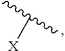

“X” represents a molecule or compound, the symbol “ [symbol] ” denotes a point of attachment of one or more chemical moieties, one or more functional groups, one or more atoms, one or more ions, an unpaired electron, or one or more other chemical species to X (where X corresponds to the represented molecule, compound, or chemical formula) via covalent bonding, wherein the covalent bonding can be any feasible covalent bond, including, but not limited to, a single bond, a double bond, or a triple bond. As an illustrative example, in the moiety

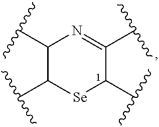 [SMILES: *C1=NC(*)C(*)[Se]C1*]

the carbon labeled “1” has point of attachment which can be a double bond to another species, such a double bond to an oxygen, or two single bonds to two independent species, such as two distinct single bonds each to a hydrogen. As another illustrative example, when two points of attachment are shown on a single atom of a molecule, such as in the moiety

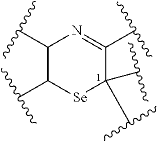 [SMILES: *C1=NC(*)C(*)[Se]C1(*)*]

where the carbon labeled “1” has two points of attachment shown, the shown points of attachment on the same single atom (e.g., carbon 1), can be interpreted as representing either a preferable embodiment of two distinct bonds to that same single atom (e.g., two hydrogens bonded to carbon 1) or an optional embodiment of a single point of attachment to said same single atom (e.g., the two points of attachment on carbon 1 can optionally be consolidated as representing one double to carbon 1, such as a double bond to oxygen). As used herein, the various functional groups represented will be understood to have a point of attachment at the functional group having the hyphen or dash (−) or a dash used in combination with an asterisk (*). In other words, in the case of —CH₂CH₂CH₃ or —CH₂CH₂CH₃, it will be understood that the point of attachment is the CH₂ group at the far left. If a group is recited without an asterisk or a dash, then the attachment point is indicated by the plain and ordinary meaning of the recited group.

Where substituent groups are specified by their conventional chemical formulae, written from left to right, they equally encompass the chemically identical substituents that would result from writing the structure from right to left, e.g., —CH₂0- is equivalent to —OCH₂—.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value. The term “substantially” refers to a property, condition, or value that is within 20%, 10%, within 5%, within 1%, optionally within 0.1%, or is equivalent to a reference property, condition, or value. The term “substantially equal”, “substantially equivalent”, or “substantially unchanged”, when used in conjunction with a reference value describing a property or condition, refers to a value that is within 20%, within 10%, optionally within 5%, optionally within 1%, optionally within 0.1%, or optionally is equivalent to the provided reference value. For example, a diameter is substantially equal to 100 nm (or, “is substantially 100 nm”) if the value of the diameter is within 20%, optionally within 10%, optionally within 5%, optionally within 1%, within 0.1%, or optionally equal to 100 nm. The term “substantially greater”, when used in conjunction with a reference value describing a property or condition, refers to a value that is at least 1%, optionally at least 5%, optionally at least 10%, or optionally at least 20% greater than the provided reference value. The term “substantially less”, when used in conjunction with a reference value describing a property or condition, refers to a value that is at least 1%, optionally at least 5%, optionally at least 10%, or optionally at least 20% less than the provided reference value. As used herein, the terms “about” and “substantially” are interchangeable. For example, a particle having a size of about 1 μm is understood to have a size is within 20%, optionally within 10%, optionally within 5%, optionally within 1%, optionally within 0.1%, or optionally equal to 1 μm.

Additional useful descriptions and discussions may be found in U.S. Provisional App. No. 62/872,149, filed Jul. 9, 2019, and in International Publication No. WO 2018/175589 (PCT/US2018/023573), each of which is incorporated herein by reference in its entirety to the extent not inconsistent herewith.

In an embodiment, a composition or compound of the invention, such as a compound comprising terlipressin or analogues, derivative, variants or fragments thereof, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

Terlipressin is a cyclic dodecamer peptide (having the sequence GGGCYFQNCPKG, SEQ. ID. NO: 1) drug, prescribed for bleeding esophageal varices, septic shock, hepatorenal syndrome and management of low blood pressure. It is an analogue of a naturally occurring hormone termed antidiuretic hormone (ADH) or vasopressin. This peptide interacts with multiple receptors in the body causing narrowing of blood vessels which leads to a rise in blood pressure. It also regulates reabsorption of water in the renal medulla, preventing excessive loss of water in the urine.

In similar to other peptide drugs, the potency of terlipressin is limited due to several key obstacles. Proteins and peptides are readily digested in the human body as they are highly susceptible to serum and tissue proteases and they are rapidly cleared from the circulation, typically in a matter of minutes. Therefore, terlipressin is administered by injections every 3-4 hours in a period of up to 3 days.

Herein, we describe the design of a novel drug carrier system based on naturally evolved interactions between long-chain fatty acids (LCFAs) and Human Serum Albumin (HSA). Mono-functionalizing octadecanedioic acid (ODDA) with terlipressin produces a prodrug that is capable of binding to HSA and shows differentiated pharmacokinetics, as well as remarkable tolerability.

Applications of the compounds, compositions, and methods described herein include: medicine for bleeding esophageal varices; medicine for septic shock; medicine for hepatorenal syndrome; medicine for management of low blood pressure; and medicine for paracentesis-induced circulatory dysfunction. Advantages of the compounds, compositions, and methods described herein include: binding the peptide to HSA offers much longer circulation time than that of the free peptide; and binding the peptide to HSA offers low immunogenicity.

Exemplary peptide Synthesis, according to certain embodiments: Terlipressin was synthesized using standard Solid Phase Peptide Synthesis (SPPS) procedures on an AAPPTec Focus XC automated synthesizer. The peptide was prepared on Rink Amide MBHA resin. A typical SPPS procedure involved FMOC deprotection with 20% methylpiperidine in DMF (one 5 min deprotection followed by one 15 min deprotection), and 45 min amide couplings using 3.75 eq of the FMOC-protected, and side chain-protected amino acid, 4 eq of HBTU and 8 eq of DIPEA.

Removal of Acm protecting group from the cysteine residues and formation of the disulfide bond was done on resin by mixing it with a thallium trifluoroacetate 1.2 eq dissolved in DMF (twice for 40 min).

Monomers were prepared by amide coupling to triisopropylsilane-protected octadecanedioic acid at the N-terminus of the peptide, following the same coupling procedure described above. Following completion of the synthesis, the peptide-fatty acid (PFA) conjugate was cleaved from the resin by treatment with TFA/H2O/TIPS in a 95:2.5:2.5 ratio for 2 h. The PFA was then precipitated in cold ether and purified by RP-HPLC. The identity of the PFA was confirmed by ESI-MS and purities were verified by observation of a single peak in analytical RP-HPLC chromatograms.

The potency of terlipressin is limited due to several key obstacles. Proteins and peptides are readily digested in the human body as they are highly susceptible to serum and tissue proteases and they are rapidly cleared from the circulation due to their low molecular weight, typically in a matter of minutes. Therefore, terlipressin has to be administered by intermittent intravenous injections every 3-4 hours in a period of up to 3 days in order to stabilize the bleeding and blood pressure.

Terlipressin is the preferred medication available for several acute conditions such as for bleeding esophageal varices, septic shock and hepatorenal syndrome.

The present disclosure offers a more efficient way to deliver the drug, reduce the number of injections and shorten the overall treatment period.

The following is a listing of exemplary non-limiting embodiments corresponding to the compounds characterized by formulas (FX1) and (FX10), as well pharmaceutical compositions, pharmaceutical salts, and methods having or using said compounds.

In some embodiments of any of the foregoing related aspects and embodiments, A¹ is selected from the group consisting of a carboxylic acid group (—COOH), a carboxylate anion (—COO⁻), or a carboxylate ester (e.g., —COOR^{a}, where R^{a} is an organic group such as an alkyl or alkoxylate group). In some such embodiments, A¹ is a carboxylic acid group. In some such embodiments, A¹ is a carboxylate ester group.

In some embodiments of any of the foregoing related aspects and embodiments, X¹ is C₈₋₃₀ hydrocarbylene, which is optionally substituted. In some further embodiments, X¹ is C₁₂₋₂₂ hydrocarbylene, which is optionally substituted. In some further embodiments, X¹ is C₁₂₋₂₂ alkylene. In some further embodiments, X¹ is —(CH₂)₁₂—, —(CH₂)₁₄—, —(CH₂)₁₆—, —(CH₂)₁₈—, —(CH₂)₂₀—, or —(CH₂)₂₂—. In some other embodiments, X¹ is —(CH₂)₁₆—. In some further embodiments, X¹ is C₁₂₋₂₂ alkenylene. In some further such embodiments, X¹ is

—(CH₂)₇—CH═CH—(CH₂)₇—.

In some further embodiments of any of the foregoing related aspects or embodiments, X¹ is C₁₂₋₂₂ hydrocarbylene, which is optionally substituted. In some such embodiments, X¹ is C₁₂₋₂₂ hydrocarbylene. In some further such embodiments, X¹ is C₁₄₋₂₂ hydrocarbylene. In some further such embodiments, X¹ is C₁₆₋₂₂ hydrocarbylene. In some embodiments of any of the aforementioned embodiments, X¹ is C₁₂₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further such embodiments, X¹ is C₁₄₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further such embodiments, X¹ is C₁₆₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further embodiments of any of the aforementioned embodiments, X¹ is C₁₂₋₂₂ straight-chain alkylene, or C₁₄₋₂₂ straight-chain alkylene, or C₁₆₋₂₂ straight-chain alkylene. In some further embodiments of any of the aforementioned embodiments, X¹ is C₁₂₋₂₂ straight-chain alkenylene, or C₁₄₋₂₂ straight-chain alkenylene, or C₁₆₋₂₂ straight-chain alkenylene.

In some embodiments of any of the foregoing related aspects and embodiments, X² is a direct bond. In some other embodiments of any of the foregoing related aspects and embodiments, X² is an organic group. In some embodiments, X² is a hydrophilic group. In some embodiments, X² is a heteroalkylene group.

In any of the aforementioned embodiments where X² is an organic group, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In any of the aforementioned embodiments where X² is a heteroalkylene group, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In some of the aforementioned embodiments, X² can contain certain groups. Some non-limiting examples of such groups that X² can contain are polyalkylene oxide groups, such as polyethylene glycol (PEG) and various polypeptide chains.

In some embodiments, X² is an organic group selected from the group consisting of —C(═O)—, —C≡C—, —C(H)═C(H)—, —C(═O)—O—, —O—C(═O)—, —C(═O)—NH—, —NH—C(═O)—, —NH—C(═O)—O—, —O—(C═O)—NH—, —O—C(═O)—O—, —C(═N—NH₂)—, —C(═N—R^{b})— (where R^{b} is a hydrogen atom or an alkyl group), —C(═N—OH)—, —NH—C(═O)—NH—, —NH—C(═S)—NH—, —NH—C(═S)—O—, —O—C(═S)—NH—, —NH—C(═O)—S—, —S—C(═O)—NH—, —NH—C(═S)—S—, —S—C(═S)—NH—, the cyclic structures shown below, and any combination thereof. In some embodiments, X² is an organic group selected from the group consisting of —C(═O)—, —C≡C—, —C(H)═C(H)—, —C(═O)—O—, —O—C(═O)—, —C(═O)—NH—, —NH—C(═O)—, —NH—C(═O)—O—, —O—(C═O)—NH—, —O—C(═O)—O—, —C(═N—NH₂)—, —C(═N—R^{b})— (where R^{b} is a hydrogen atom or an alkyl group), —C(═N—OH)—, —NH—C(═O)—NH—, —NH—C(═S)—NH—, —NH—C(═S)—O—, —O—C(═S)—NH—, —NH—C(═O)—S—, —S—C(═O)—NH—, —NH—C(═S)—S—, —S—C(═S)—NH—, and the cyclic structures shown below:

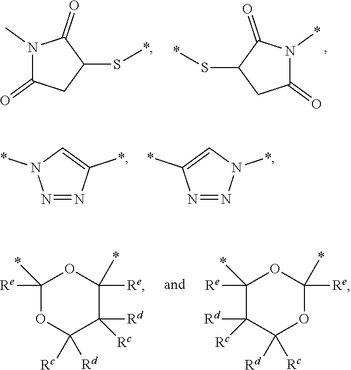

where R^{c}, R^{d}, and R^{e} are, independently at each occurrence, a hydrogen atom or C₁-10 alkyl. In some further embodiments, X² is —C(═O)—.

In some embodiments, X² is a group selected from the group consisting of —O—, —S—, —S(═O)—, —S(═O)₂—, —S—S—, —N═, ═N—, —N(H)—, —N═N—N(H)—, —N(H)—N═N—, —N(OH)—, and —N(O)—.

In some embodiments, X² comprises one or more moieties selected from the group consisting of: —C(═O)—, —O—C(═O)—, —NH—C(═O)—, one or more moieties formed from a alkylene glycols, one or more units formed from alkanol amines, one or more units formed from amino acids, and one or more units formed from hydroxyl acids. Thus, in some embodiments, X² comprises one or more moieties formed from alkylene glycols, such as a short poly(ethylene glycol) chain having 1 to 25 ethylene glycol units. In some embodiments, X² comprises one or more moieties formed from amino acids, such as an oligopeptide chain having 1 to 25 amino acid units. In some embodiments, X² comprises one or more moieties formed from hydroxy acids, such as moieties formed from glycolic acid, lactic acid, or caprolactone. In some embodiments, X² comprises a combination of a poly(ethylene glycol) chain having 1 to 25 ethylene glycol units and an oligopeptide having 1 to 25 amino acid units, and optionally one or more units formed from hydroxy acids.

In any of the above embodiments, the selection of X² can depend on the type of functional group through which it is linked to the peptide moiety A², so as to avoid making compounds that are chemically unstable or impossible. The skilled artisan will be able to select combinations of X² and A² that result in chemically stable compounds. Chemically stable compounds are optionally those of which the chemical structure is not substantially altered when kept at a temperature from about −80° C. to about +40° C., in the absence of moisture or other chemically reactive conditions, for at least a week.

The selection of —X²—X¹-A¹ can depend on the nature of the connection to the drug moiety. For example, in embodiments where the —X²—X¹-A¹ connects to an oxygen atom or an NH group on the A² peptide moiety, as is the case of conjugation to the N-terminus of the protein in Table 1, then —X²—X¹-A¹ can be: —C(═O)—(CH₂)ₙ₁—C(═O)—OH; —C(═O)—(CH₂)ₙ₁—C(═O)—OCH₃; —C(═O)—(CH₂)ₙ₁—CH₃; —C(═O)—(C₁₋₆ alkylene)-C(═O)—O—(CH₂)ₙ₂—C(═O)—OH; —C(═O)—(C₁₋₆ alkylene)-NH—C(═O)—(CH₂)ₙ₁—C(═O)—OH; —C(═O)—(C₁₋₆ alkylene)-C(═O)—O—[(CH₂)₂—O-]ₙ₃(CH₂)ₙ₂—C(═O)—OH; —C(═O)—O—(CH₂)ₙ₂—C(═O)—OH; and —C(═O)—NH—(CH₂)ₙ₂—C(═O)—OH; wherein n1 is an integer 12 to 24, n2 is an integer from 13 to 25, and n3 is an integer from 1 to 25. In some further such embodiments, —X²—X¹-A¹ is selected from the group consisting of: —C(═O)—(CH₂)ₙ₁—C(═O)—OH; —C(═O)—(CH₂)ₙ₁—C(═O)—OCH₃; —C(═O)—(C₁₋₆ alkylene)-C(═O)—O—(CH₂)ₙ₂—C(═O)—OH; —C(═O)—(C₁₋₆ alkylene)-NH—C(═O)—(CH₂)ₙ₁—C(═O)—OH; —C(═O)—(C₁₋₆ alkylene)-C(═O)—O—[(CH₂)₂—O-]ₙ₃(CH₂)ₙ₂—C(═O)—OH; —C(═O)—O—(CH₂)ₙ₂—C(═O)—OH; or —C(═O)—NH—(CH₂)ₙ₂—C(═O)—OH. In some further such embodiments, —X²—X¹-A¹ can be: —C(═O)—(CH₂)ₙ₁—C(═O)—OH; —C(═O)—O—(CH₂)ₙ₂—C(═O)—OH; or —C(═O)—NH—(CH₂)ₙ₂—C(═O)—OH. In some other embodiments, —X²—X¹-A¹ is —C(═O)—(C₁₋₆ alkylene)-O—C(═O)—(CH₂)ₙ₁—C(═O)—OH, where n1 is an integer from 12 to 24. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, —X²—X¹-A¹ is —C(═O)—(C₁₋₆ alkylene)-C(═O)—O—(CH₂)ₙ₃—OH, where n3 is an integer from 14 to 26, or an integer from 16 to 24, or an integer from 18 to 22.

In embodiments where the —X²—X¹-A¹ connects to a —C(═O) group on the drug moiety, such as on the C-terminus of the peptide moieties recited in Table 1, then —X²—X¹-A¹ can be: —O—(CH₂)ₙ₂—C(═O)—OH; —NH—(CH₂)ₙ₂—C(═O)—OH; —NH—(C₁₋₆ alkylene)-O—C(═O)—(CH₂)ₙ₁—C(═O)—OH; —O—(C₁₋₆ alkylene)-O—C(═O)—(CH₂)ₙ₁—C(═O)—OH; —NH—(C₁₋₆ alkylene)-O—C(═O)—(CH₂)ₙ₁—C(═O)—OCH₃; —O—(C₁₋₆ alkylene)-O—C(═O)—(CH₂)ₙ₁—C(═O)—OCH₃; —NH—(C₁₋₆ alkylene)-O—C(═O)—(CH₂)ₙ₁—CH₃; —O—(C₁₋₆ alkylene)-O—C(═O)—(CH₂)ₙ₁—CH₃; —NH—(C₁₋₆ alkylene)-C(═O)—O—[(CH₂)₂—O-]ₙ₃(CH₂)ₙ₂—C(═O)—OH; or —O—(C₁₋₆ alkylene)-C(═O)—O—[(CH₂)₂—O-]ₙ₃(CH₂)ₙ₁—C(═O)—OH; wherein n1 is an integer 12 to 24, n2 is an integer from 13 to 25, and n3 is an integer from 1 to 25. In some further such embodiments, —X²—X¹-A¹ can be: —O—(CH₂)ₙ₂—C(═O)—OH; —NH—(CH₂)ₙ₂—C(═O)—OH; —NH—(C₁₋₆ alkylene)-O—C(═O)—(CH₂)ₙ₁—C(═O)—OH; —O—(C₁₋₆ alkylene)-O—C(═O)—(CH₂)ₙ₁—C(═O)—OH; —NH—(C₁₋₆ alkylene)-O—C(═O)—(CH₂)ₙ₁—C(═O)—OCH₃; or —O—(C₁₋₆ alkylene)-O—C(═O)—(CH₂)ₙ₁—C(═O)—OCH₃. In some further such embodiments, —X²—X¹-A¹ can be: —O—(CH₂)ₙ₂—C(═O)—OH; —NH—(CH₂)ₙ₂—C(═O)—OH; —NH—(C₁₋₆ alkylene)-O—C(═O)—(CH₂)ₙ₁—C(═O)—OH; or —O—(C₁₋₆ alkylene)-O—C(═O)—(CH₂)ₙ₁—C(═O)—OH. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, —X²—X¹-A¹ is —O—(CH₂)ₙ₃—OH, where n3 is an integer from 14 to 26, or an integer from 16 to 24, or an integer from 18 to 22.

The compounds described in any of the above embodiments can also exist as pharmaceutically acceptable salts. The term “pharmaceutically acceptable salts” refers to salts of the compounds which are not biologically or otherwise undesirable and are generally prepared by reacting the free base with a suitable organic or inorganic acid or by reacting the acid with a suitable organic or inorganic base. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, there can be formed an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate, and the like.

The compounds above can be made by standard organic synthetic methods, such as those illustrated in: Wuts et al., Greene's Protective Groups in Organic Synthesis (4th ed., 2006); Larock, Comprehensive Organic Transformations (2nd ed., 1999); and Smith et al, March's Advanced Organic Chemistry: Reactions, Mechanisms, and Structure (6th ed., 2007); Methods of Molecular Biology, 35, Peptide Synthesis Protocols, (M. W. Pennington and B. M. Dunn Eds), Springer, 1994; Methods of Enzymology, 289, Solid Phase Peptide Synthesis, (G. B. Fields Ed.), Academic Press, 1997; Chemical Approaches to the Synthesis of Peptides and Proteins, (P. Lloyd-Williams, F. Albericio, and E. Giralt Eds), CRC Press, 1997; Fmoc Solid Phase Peptide Synthesis, A Practical Approach, (W. C. Chan, P. D. White Eds), Oxford University Press, 2000; Solid Phase Synthesis, A Practical Guide, (S. F. Kates, F Albericio Eds), Marcel Dekker, 2000; P. Seneci, Solid-Phase Synthesis and Combinatorial Technologies, John Wiley & Sons, 2000; Synthesis of Peptides and Peptidomimetics (M. Goodman, Editor-in-chief, A. Felix, L. Moroder, C. Tmiolo Eds), Thieme, 2002; N. L. Benoiton, Chemistry of Peptide Synthesis, CRC Press, 2005. Specific non-limiting examples are shown below in the Examples.

The compounds of the foregoing embodiments, including their pharmaceutically acceptable salts, are useful as compounds for the treatment of, for example, hepatorenal syndrome, low blood pressure, bleeding esophageal varices, septic shock paracentesis-induced circulatory dysfunction, a condition or disease that can be treated using vasoconstriction or albumin-mediated vasoconstriction, and any combination thereof.

Table 1 shows illustrative exemplary, non-limiting, moieties for the A²-peptide moiety, wherein A² can be the moiety shown (terlipressin), a pharmaceutically acceptable salt thereof, a substituted or unsubstituted derivative thereof, a substituted or unsubstituted natural or synthetic analogue thereof, a substituted or unsubstituted variant thereof, a substituted or unsubstituted isomer thereof, a substituted or unsubstituted fragment thereof, or a peptide moiety comprising a sequence having 80% or greater sequence homology therewith. Table 2 shows illustrative exemplary, non-limiting, moieties for —X²—X¹-A¹. Table 3 refers to various combinations of an A²-moiety with a —X²—X¹-A¹, which together form certain exemplary, non-limiting, compounds of the present disclosure. Table 3 shows non-limiting illustrative combinations of the moieties from Tables 1 and 2, which can come together to form certain exemplary compounds according to the present disclosure. Also disclosed herein are compounds including the A² peptide moiety according to Table 1, or any peptide moiety having an 80% or greater sequence homology of Seq. ID. 1 (GGGCYFQNCPKG), combined with (conjugated with) any of the —X²—X¹-A¹ moieties shown in Table 2, wherein such combination is not sterncally impractical and/or synthetically non-feasible. In some embodiments, also disclosed herein are compounds including the A² peptide moiety according to Table 1, or any peptide moiety having an 80% or greater sequence homology of Seq. ID. 1 (GGGCYFQNCPKG), combined with (conjugated with) more than one (e.g., up to 10) of any of the —X²—X¹-A¹ moieties shown in Table 2, wherein such combination is not sterncally impractical and/or synthetically non-feasible. The compounds disclosed herein, including those in Table 3, can be made by methods analogous to those illustrated in the Examples, and by common synthetic methods known to those of ordinary skill in the art. Suitable methods of making such compounds are illustrated in: Wuts et al., Greene's Protective Groups in Organic Synthesis (4th ed., 2006); Larock, Comprehensive Organic Transformations (2nd ed., 1999); and Smith et al., March's Advanced Organic Chemistry: Reactions, Mechanisms, and Structure (6th ed., 2007).

TABLE 1

| | A²-Moieties |
|---|---|
| HA1 | GGGCYFQNCPKG (SEQ. ID. NO: 1) |

TABLE 2

| | —X²—X¹—A¹ Moieties |
|---|---|
| HB1 | —C(═O)—(CH₂)₁₄—C(═O)—OH |
| HB2 | —C(═O)—(CH₂)₁₆—C(═O)—OH |
| HB3 | —C(═O)—(CH₂)₁₈—C(═O)—OH |
| HB4 | —C(═O)—(CH₂)₇—CH═CH—(CH₂)₇—C(═O)—OH |
| HB5 | —C(═O)—(CH₂)₁₄—C(═O)—O—CH₃ |
| HB6 | —C(═O)—(CH₂)₁₆—C(═O)—O—CH₃ |
| HB7 | —C(═O)—(CH₂)₁₈—C(═O)—O—CH₃ |
| HB8 | —C(═O)—(CH₂)₇—CH═CH—(CH₂)₇—C(═O)—O—CH₃ |
| HB9 | —C(═O)—(CH₂)₁₄—CH₃ |
| HB10 | —C(═O)—(CH₂)₁₆—CH₃ |
| HB11 | —C(═O)—(CH₂)₁₈—CH₃ |
| HB12 | —C(═O)—(CH₂)₇—CH═CH—(CH₂)₇—CH₃ |
| HB13 | —C(═O)—(CH₂)₂—C(═O)—O—(CH₂)₁₅—C(═O)—OH |
| HB14 | —C(═O)—(CH₂)₂—C(═O)—O—(CH₂)₁₇—C(═O)—OH |
| HB15 | —C(═O)—(CH₂)₂—C(═O)—O—(CH₂)₁₉—C(═O)—OH |
| HB16 | —C(═O)—(CH₂)₂—C(═O)—O—(CH₂)₈—CH═CH—(CH₂)₇—C(═O)—OH |
| HB17 | —C(═O)—CH₂—NH—C(═O)—(CH₂)₁₄—C(═O)—OH |
| HB18 | —C(═O)—CH₂—NH—C(═O)—(CH₂)₁₆—C(═O)—OH |
| HB19 | —C(═O)—CH₂—NH—C(═O)—(CH₂)₁₈—C(═O)—OH |
| HB20 | —C(═O)—CH₂—NH—C(═O)—(CH₂)₇—CH═CH—(CH₂)₇—C(═O)—OH |
| HB21 | —C(═O)—(CH₂)₂—C(═O)—O—[(CH₂)₂—O—]₆C(═O)—(CH₂)₁₄—C(═O)—OH |
| HB22 | —C(═O)—(CH₂)₂—C(═O)—O—[(CH₂)₂—O—]₆C(═O)—(CH₂)₁₆—C(═O)—OH |
| HB23 | —C(═O)—(CH₂)₂—C(═O)—O—[(CH₂)₂—O—]₆C(═O)—(CH₂)₁₈—C(═O)—OH |
| HB24 | —C(═O)—(CH₂)₂—C(═O)—O—[(CH₂)₂—O—]₆C(═O)—(CH₂)₇—CH═CH—(CH₂)₇—C(═O)—OH |
| HB25 | —C(═O)—O—(CH₂)₁₅—C(═O)—OH |
| HB26 | —C(═O)—O—(CH₂)₁₇—C(═O)—OH |
| HB27 | —C(═O)—O—(CH₂)₁₉—C(═O)—OH |
| HB28 | —C(═O)—O—(CH₂)₈—CH═CH—(CH₂)₇—C(═O)—OH |
| HB29 | —C(═O)—NH—(CH₂)₁₅—C(═O)—OH |
| HB30 | —C(═O)—NH—(CH₂)₁₇—C(═O)—OH |
| HB31 | —C(═O)—NH—(CH₂)₁₉—C(═O)—OH |
| HB32 | —C(═O)—NH—(CH₂)₈—CH═CH—(CH₂)₇—C(═O)—OH |
| HB33 | —O—(CH₂)₁₅—C(═O)—OH |
| HB34 | —O—(CH₂)₁₇—C(═O)—OH |
| HB35 | —O—(CH₂)₁₉—C(═O)—OH |
| HB36 | —O—(CH₂)₈—CH═CH—(CH₂)₇—C(═O)—OH |
| HB37 | —NH—(CH₂)₂—O—C(═O)—(CH₂)₁₄—C(═O)—OH |
| HB38 | —NH—(CH₂)₂—O—C(═O)—(CH₂)₁₆—C(═O)—OH |
| HB39 | —NH—(CH₂)₂—O—C(═O)—(CH₂)₁₈—C(═O)—OH |
| HB40 | —NH—(CH₂)₂—O—C(═O)—(CH₂)₇—CH═CH—(CH₂)₇—C(═O)—OH |
| HB41 | —O—(CH₂)₂—O—C(═O)—(CH₂)₁₄—C(═O)—OH |
| HB42 | —O—(CH₂)₂—O—C(═O)—(CH₂)₁₆—C(═O)—OH |
| HB43 | —O—(CH₂)₂—O—C(═O)—(CH₂)₁₈—C(═O)—OH |
| HB44 | —O—(CH₂)₂—O—C(═O)—(CH₂)₇—CH═CH—(CH₂)₇—C(═O)—OH |
| HB45 | —NH—CH₂—C(═O)—O—[(CH₂)₂—O—]₆C(═O)—(CH₂)₁₄—C(═O)—OH |
| HB46 | —NH—CH₂—C(═O)—O—[(CH₂)₂—O—]₆C(═O)—(CH₂)₁₆—C(═O)—OH |
| HB47 | —NH—CH₂—C(═O)—O—[(CH₂)₂—O—]₆C(═O)—(CH₂)₁₈—C(═O)—OH |
| HB48 | —NH—CH₂—C(═O)—O—[(CH₂)₂—O—]₆C(═O)—(CH₂)₇—CH═CH—(CH₂)₇—C(═O)—OH |
| HB49 | —NH—(CH₂)₂—O—C(═O)—(CH₂)₁₄—C(═O)—O—CH₃ |
| HB50 | —NH—(CH₂)₂—O—C(═O)—(CH₂)₁₆—C(═O)—O—CH₃ |
| HB51 | —NH—(CH₂)₂—O—C(═O)—(CH₂)₁₈—C(═O)—O—CH₃ |
| HB52 | —NH—(CH₂)₂—O—C(═O)—(CH₂)₇—CH═CH—(CH₂)₇—C(═O)—O—CH₃ |
| HB53 | —CH₂—O—C(═O)—(CH₂)₁₄—C(═O)—OH |
| HB54 | —CH₂—O—C(═O)—(CH₂)₁₆—C(═O)—OH |
| HB55 | —CH₂—O—C(═O)—(CH₂)₁₈—C(═O)—OH |
| HB56 | —CH₂—O—C(═O)—(CH₂)₇—CH═CH—(CH₂)₇—C(═O)—OH |
| HB57 | —CH₂—O—C(═O)—(CH₂)₁₄—C(═O)—O—CH₃ |
| HB58 | —CH₂—O—C(═O)—(CH₂)₁₆—C(═O)—O—CH₃ |
| HB59 | —CH₂—O—C(═O)—(CH₂)₁₈—C(═O)—O—CH₃ |
| HB60 | —CH₂—O—C(═O)—(CH₂)₇—CH═CH—(CH₂)₇—C(═O)—O—CH₃ |
| HB61 | —CH₂—O—C(═O)—(CH₂)₁₄—CH₃ |
| HB62 | —CH₂—O—C(═O)—(CH₂)₁₆—CH₃ |
| HB63 | —CH₂—O—C(═O)—(CH₂)₁₈—CH₃ |
| HB64 | —CH₂—O—C(═O)—(CH₂)₇—CH═CH—(CH₂)₇—CH₃ |
| HB65 | —CH₂—O—C(═O)—CH₂—NH—C(═O)—(CH₂)₁₄—C(═O)—OH |
| HB66 | —CH₂—O—C(═O)—CH₂—NH—C(═O)—(CH₂)₁₆—C(═O)—OH |
| HB67 | —CH₂—O—C(═O)—CH₂—NH—C(═O)—(CH₂)₁₈—C(═O)—OH |
| HB68 | —CH₂—O—C(═O)—CH₂—NH—C(═O)—(CH₂)₇—CH═CH—(CH₂)₇—C(═O)—OH |
| HB69 | —CH₂—O—C(═O)—(CH₂)₂—C(═O)—O—[(CH₂)₂—O—]₆C(═O)—(CH₂)₁₄—C(═O)—OH |
| HB70 | —CH₂—O—C(═O)—(CH₂)₂—C(═O)—O—[(CH₂)₂—O—]₆C(═O)—(CH₂)₁₆—C(═O)—OH |
| HB71 | —CH₂—O—C(═O)—(CH₂)₂—C(═O)—O—[(CH₂)₂—O—]₆C(═O)—(CH₂)₁₈—C(═O)—OH |
| HB72 | —CH₂—O—C(═O)—(CH₂)₂—C(═O)—O—[(CH₂)₂—O—]₆C(═O)—(CH₂)₇—CH═CH—(CH₂)₇—C(═O)—OH |
| HB73 | —CH₂—O—C(═O)—O—(CH₂)₁₅—C(═O)—OH |
| HB74 | —CH₂—O—C(═O)—O—(CH₂)₁₇—C(═O)—OH |
| HB75 | —CH₂—O—C(═O)—O—(CH₂)₁₉—C(═O)—OH |
| HB76 | —CH₂—O—C(═O)—O—(CH₂)₈—CH═CH—(CH₂)₇—C(═O)—OH |
| HB77 | —CH₂—O—C(═O)—NH—(CH₂)₁₅—C(═O)—OH |
| HB78 | —CH₂—O—C(═O)—NH—(CH₂)₁₇—C(═O)—OH |
| HB79 | —CH₂—O—C(═O)—NH—(CH₂)₁₉—C(═O)—OH |
| HB80 | —CH₂—O—C(═O)—NH—(CH₂)₈—CH═CH—(CH₂)₇—C(═O)—OH |
| HB81 | ═N—O—(CH₂)₁₅—C(═O)—OH |
| HB82 | ═N—O—(CH₂)₁₇—C(═O)—OH |
| HB83 | ═N—O—(CH₂)₁₉—C(═O)—OH |
| HB84 | ═N—O—(CH₂)₈—CH═CH—(CH₂)₇—C(═O)—OH |
| HB85 | ═N—NH—(CH₂)₁₅—C(═O)—OH |
| HB86 | ═N—NH—(CH₂)₁₇—C(═O)—OH |
| HB87 | ═N—NH—(CH₂)₁₉—C(═O)—OH |
| HB88 | ═N—NH—(CH₂)₈—CH═CH—(CH₂)₇—C(═O)—OH |
| HB89 | ═N—O—[(CH₂)₂—O—]₆(CH₂)₁₅—C(═O)—OH |
| HB90 | ═N—O—[(CH₂)₂—O—]₆(CH₂)₁₇—C(═O)—OH |
| HB91 | ═N—O—[(CH₂)₂—O—]₆(CH₂)₁₉—C(═O)—OH |
| HB92 | ═N—O—[(CH₂)₂—O—]₆(CH₂)₈—CH═CH—(CH₂)₇—C(═O)—OH |
| HB93 | —C(═O)—CH₂—O—C(═O)—(CH₂)₁₄—C(═O)—OH |
| HB94 | —C(═O)—CH₂—O—C(═O)—(CH₂)₁₆—C(═O)—OH |
| HB95 | —C(═O)—CH₂—O—C(═O)—(CH₂)₁₈—C(═O)—OH |
| HB96 | —C(═O)—CH₂—O—C(═O)—(CH₂)₇—CH═CH—(CH₂)₇—C(═O)—OH |
| HB97 | —C(═O)—CH(CH₃)—O—C(═O)—(CH₂)₁₄—C(═O)—OH |
| HB98 | —C(═O)—CH(CH₃)—O—C(═O)—(CH₂)₁₆—C(═O)—OH |
| HB99 | —C(═O)—CH(CH₃)—O—C(═O)—(CH₂)₁₈—C(═O)—OH |
| HB100 | —C(═O)—CH(CH₃)—O—C(═O)—(CH₂)₇—CH═CH—(CH₂)₇—C(═O)—OH |
| HB101 | —C(═O)—(CH₂)₅—O—C(═O)—(CH₂)₁₄—C(═O)—OH |
| HB102 | —C(═O)—(CH₂)₅—O—C(═O)—(CH₂)₁₆—C(═O)—OH |
| HB103 | —C(═O)—(CH₂)₅—O—C(═O)—(CH₂)₁₈—C(═O)—OH |
| HB104 | —C(═O)—(CH₂)₅—O—C(═O)—(CH₂)₇—CH═CH—(CH₂)₇—C(═O)—OH |

TABLE 3

| A²-Moiety | —X²—X¹—A¹ Moiety |
|---|---|
| HA1 | HB1, HB2, HB3, HB4, HB5, HB6, HB7, HB8, HB9, |
| | HB10, HB11, HB12, HB13, HB14, HB15, HB16, |
| | HB17, HB18, HB19, HB20, HB21, HB22, HB23, |
| | HB24, HB25, HB26, HB27, HB28, HB29, HB30, |
| | HB31, HB32, HB33, HB34, HB35, HB36, HB37, |
| | HB38, HB39, HB40, HB41, HB42, HB43, HB44, |
| | HB45, HB46, HB47, HB46, HB49, HB50, HB51, |
| | HB52, HB53, HB54, HB55, HB56, HB57, HB58, |
| | HB59, HB60, HB61, HB62, HB63, HB64, HB65, |
| | HB66, HB67, HB68, HB69, HB70, HB71, HB72, |
| | HB73, HB74, HB75, HB76, HB77, HB78, HB79, |
| | HB80, HB81, HB82, HB83, HB84, HB85, HB86, |
| | HB87, HB88, HB89, HB90, HB91, HB92, HB93, |
| | HB94, HB95, HB96, HB97, HB98, HB99, HB100, |
| | HB101, HB102, HB103, HB104, respectively |

The invention can be further understood by the following non-limiting examples.

### Example 1: Terlipressin-Octadecanedioic Acid Conjugate for Albumin-Mediated Vasoconstrictive Therapy

Hepatorenal syndrome (HRS) is a serious complication of liver cirrhosis with critically poor prognosis and no approved medication in the U.S. We are developing a novel therapeutic agent by the synthesis of an octadecanedioic diacid (ODDA) conjugate of a potent vasoconstrictor peptide drug termed terlipressin. Studies show that the conjugate can stimulate vasocontraction in cultured smooth muscle cells.

Proposed studies include pharmacokinetic investigation and direct measurement of the vasoconstrictive effect using radiotelemetry, as well as measurements of the antidiuretic effect of the drug in an animal model. Successful completion of the project will lead to a new life-saving therapy to benefit patients, allowing an immediate and easily delivered treatment without the need of a specialized physician, sophisticated equipment or surgery.

Hepatorenal syndrome (HRS) is the most frequent life-threatening complication of advanced liver failure and cirrhosis.¹ The number of adults with diagnosed chronic liver disease in the US is estimated at 3.9 million. HRS results from a functional renal dysfunction due to circulatory disturbances in patients with decompensated liver cirrhosis, acute liver failure or alcoholic hepatitis. The prognosis for HRS is very poor with expected survival time of up to 6 months from diagnosis. HRS also imposes a significant healthcare burden (˜$3.5 billion to US tax payers annually).² Liver transplantation is considered the treatment of choice for patients with cirrhosis and HRS, but many patients do not survive long enough to receive a transplant and currently available medical therapies are ineffective in a substantial proportion of patients. Moreover, many of these patients do not qualify for transplant because they are alcoholics.

Terlipressin is a synthetic short peptide, analogue of natural hormone vasopressin, which is being used as standard therapy for acute variceal hemorrhage HRS in Europe, Asia and Australia, but not in the U.S.²⁻⁵ Treatment with terlipressin increases mean arterial pressure and decreases portal flow and pressure within minutes of administration. Moreover, Treatment with terlipressin was shown to be considerably safe and improve the survival rate.⁴

Despite the attractive pharmacological profile of terlipressin, in similar fashion to most peptide-based drugs, key issues limit its clinical utility and have prevented approval in the US to date. Mainly, this rests on its susceptibility to proteolytic enzymes, and rapid, size-dependent excretion by the kidneys.⁶⁻⁹ As a result, terlipressin suffers from poor half-life of less than an hour. Consequently, the drug has to be administered by intermittent injections in frequency of 4-6 hours for a minimum of 2 consecutive days and up to several weeks,⁴ causing great discomfort to the patients and requiring an excessive amount of material delivered in the clinic by experts.

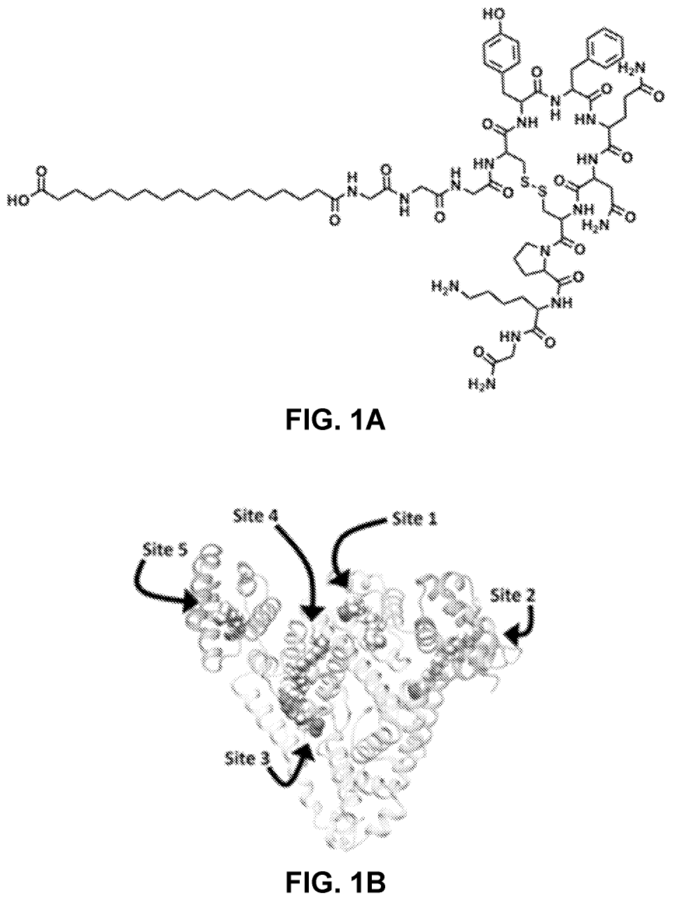

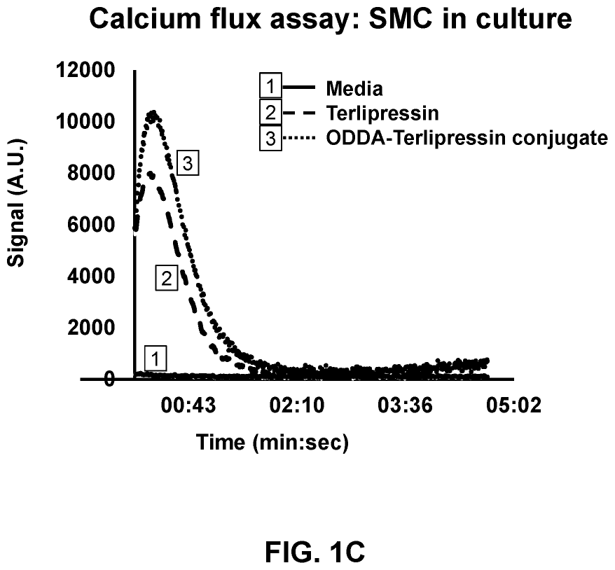

Exemplary experimental procedures: Solutions to the limitations of native peptides have been proposed including modification of the peptide sequence with unnatural amino acids, peptide stapling or oligomerization.¹⁰ However, few of these strategies can be readily generalized and none has been demonstrated as a promising solution to both problems. We have developed a system based on naturally evolved interactions between long-chain fatty acids and the protein human serum albumin (HSA) (FIG. 1B). Specifically, mono-functionalizing octadecanedioic acid with a drug, has proven a powerful and potentially generalizable strategy for extension of half-life. Indeed, we have recently published on this strategy, for the delivery of chemotherapeutics.¹¹ It must be noted that, the first and only example of a drug using a long-chain fatty diacid is Ozempic (semaglutide). This ODDA-peptide conjugate was approved by the FDA in 2017. Our hypothesis is that by maintaining the naturally evolved LCFA carboxylate contact one engages HSA's natural binding site. This is a critical feature contributing to semaglutide's performance as a once-weekly glucagon-like-peptide-1 receptor agonist for type-2 diabetes. Similarly, we contemplate extension of half-life of other peptide drugs will be a fruitful strategy to invigorating peptide-drug development and delivery in general. Terlipressin produces a prodrug that is capable of binding HSA which offers low immunogenicity and far longer circulation time than that of the free peptide.¹² Moreover, there is accumulating evidence that treatment of terlipressin in combination with albumin is more effective and strongly associated with improved survival.¹³

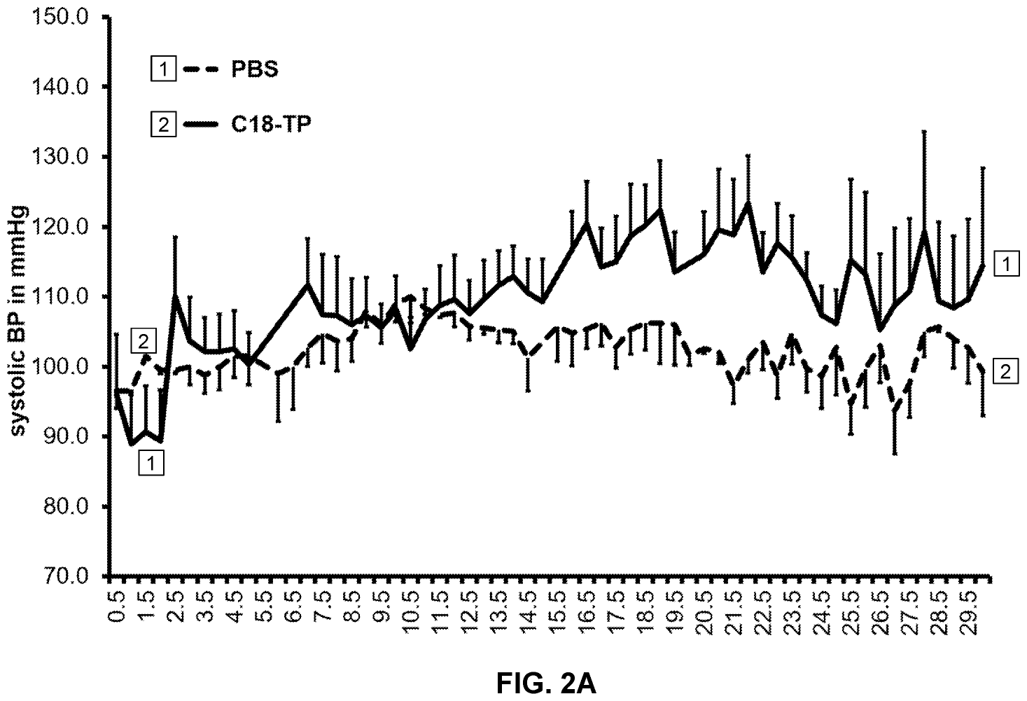

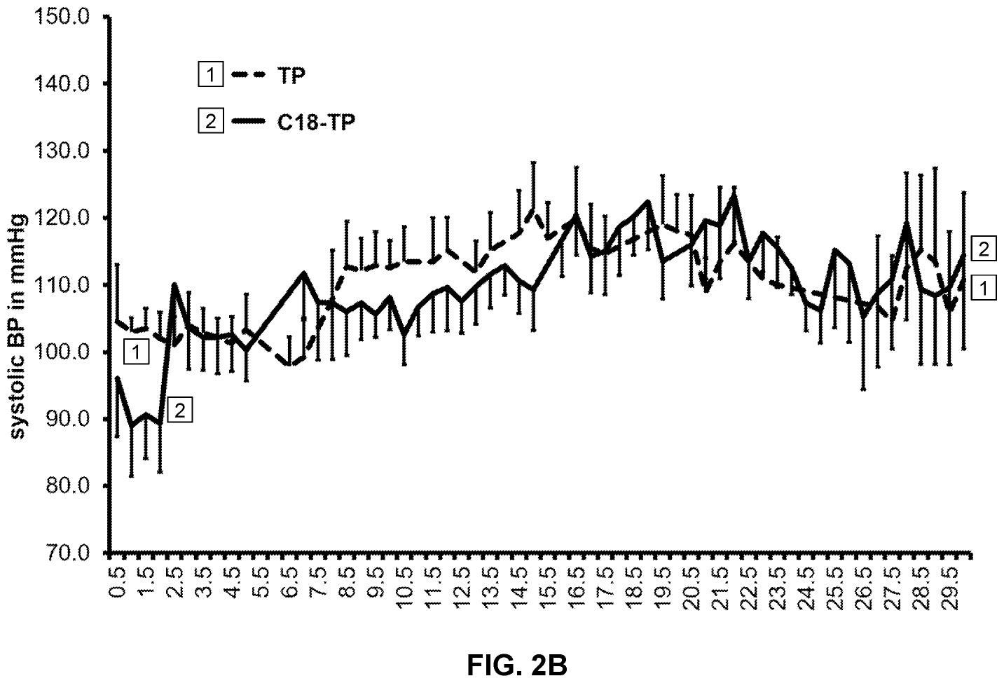

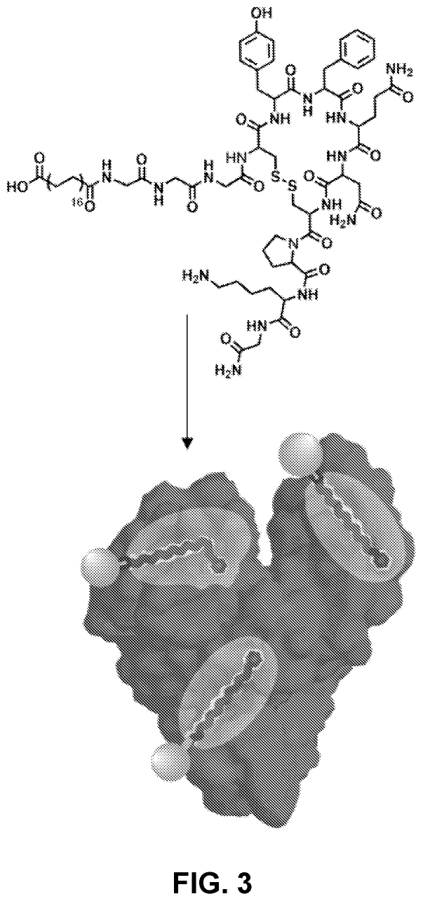

Using this system, we have demonstrated that terlipressin conjugates, synthesized in the Gianneschi lab, can bind to and activate smooth muscle cells (SMCs) in vitro as indicated by an influx of intracellular calcium (FIG. 1C). SMCs are the cellular components of the normal blood vessel wall that provide structural integrity and regulate the diameter by contracting and relaxing dynamically in response to vasoactive stimuli. Furthermore, we employed a tail-cuff system coupled with a volume pressure recording sensor to allow accurate non-invasive blood pressure measurements in mice. Terlipressin conjugates were administrated to C57BL mice by intraperitoneal injection (IP) at the Batlle lab. The C18 ODDA-terlipressin conjugates were shown to increase the systolic blood pressure of the mice, compared with negative control (vehicle) and positive control (terlipressin peptide) (FIGS. 2A-2B).

Employing this system for treatment of bleeding varices HRS instead of native terlipressin could lead to fewer injections, relieving the patients of unnecessary distress, and using much smaller amounts of material. There are currently over 50 ongoing clinical studies exploring further applications of terlipressin including hepatorenal syndrome bleeding esophageal varices, low blood pressure and hyponatremia⁴ making this peptide drug highly desirable for various medical conditions. Furthermore, successful results in this project can serve as a model for a myriad of different injectable peptide drugs that previously failed clinical trials due to the constraints mentioned above.

Medical Need: The number of adults with diagnosed chronic liver disease in the U.S. is 3.9 million. More than 90% of these patients will develop esophageal varices due to portal hypertension sometime in their lifetime, and about 30% will start bleeding.¹

Pharmacokinetic profile study: Distribution and elimination half-life of the terlipressin conjugate is measured in mice using labeled conjugates and compared with the free peptide direct LCMS quantification assays from blood samples. The conjugated terlipressin compounds are compared with the free peptide as a direct analysis of extension of half-life with our ODDA-analogue.

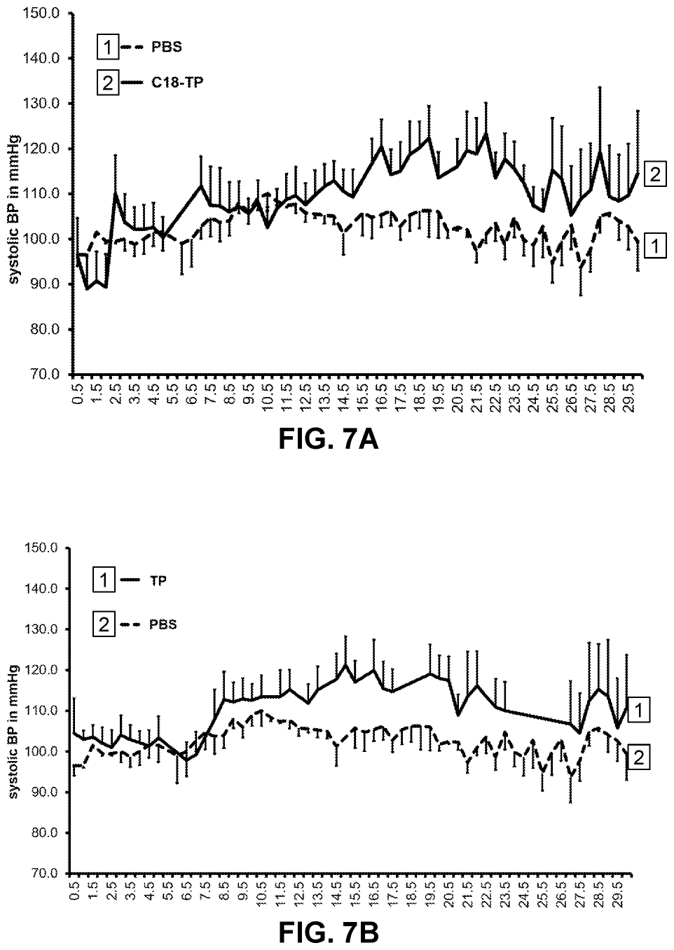

Vasoconstriction study in disease model; Blood pressure measurements: Portal and systemic circulatory effects of terlipressin conjugates are studied. Portal hypertension are induced by partial portal vein ligation in mice as described previously,¹³ and the effect of a range of doses of the free peptide and peptide conjugate on systemic and splanchnic circulation as well as portal pressure are measured through time following injection by implanting radio-telemetry probes. The effects of ODDA-TP are studied in C57-Bl mice both acutely¹⁴ (FIGS. 7A-7B) and by radiotelemetry for a week long duration of response. These are directly compared to terlipressin peptide alone.¹⁵

Antidiuretic study: Terlipressin is also involved with reabsorption of water in the kidney owing to its V2 receptor agonistic activity.¹⁶. This antidiuretic effect are studied by collection of urine from the animals following injection of peptide conjugates or free peptide and measuring changes in conductivity of urine flow.

### REFERENCES CORRESPONDING TO EXAMPLE 1

1. Cordon J P, Torres C F, Garcia A B, Rodriguez F G, J M S de Parga. World J. Gastrointest. Endosc. 4, 312-22 (2012)
2. Ioannou G N, Doust J, Rockey D C. Cochrane Database Syst. Rev. CD002147 (2003)
3. Krag A, Borup T, Moller S, Bendtsen F. Adv Ther. 25, 1105-1140 (2008)
4. Kam P C A, Williams S, Yoong F F Y. Anaesthesia 59, 993-1001 (2004)
5. Papaluca T, Gow P. J. Gastroenterol. Hepatol. 33, 591-598 (2018)
6. Craik D J, Fairlie D P, Liras S, Price D. Chem. Biol. Drug Des. 81, 136-147 (2013)
7. Kaspar A A, Reichert J M. Drug Discovery Today 18, 807-817 (2013)
8. McGregor D P. Curr. Opin. Pharmacol. 8, 616-619 (2008)
9. Vlieghe P, Lisowski V, Martinez J, Khrestchatisky M. Drug Discovery Today 15, 40-56 (2010)
10. Goodwin D, Simerska P, Toth I. Current Medicinal Chemistry 19, 4451-4461 (2012)
11. Liu Z, Chen X. Chem. Soc. Rev. 45, 1432-1456 (2016)
12. Neri S, Pulvirenti D, Malaguamera M. Cosimo B M, Bertino G, Ignaccolo L, Siringo S, Castellino P. Dig. Dis. Sci. 53, 830-835 (2008).
13. Bemadich C, Bandi J C, Melin P, Bosch J. Hepatology 27, 351-356 (1998).
1. Erly B, Carey W D, Kapoor B, McKinney J M, Tam M, Wang W. Semin Intervent Radiol. 32:4, 445-454. (2015)
2. Rice J B, White A G, Galebach P, Korenblat K M, Wagh A, Lovelace B, Wan G J, Jamil K. Current Medical Research and Opinion. 33:8, 1473-1480 (2017)
11. Callmann C E, LeGuyader C L M, Burton S T, Thompson M P, Hennis R, Barback C, Henriksen N M, Chan W C, Jaremko M J, Yang J, Garcia A, Burkart M D, Gilson M K, Momper J D, Bertin P A, Gianneschi N C. J. Am. Chem. Soc. 141, 11765-11769 (2019)
13. Neri S, Pulvirenti D, Malaguamera M. Cosimo B M, Bertino G, Ignaccolo L, Siringo S, Castellino P. Dig. Dis. Sci. 53, 830-835 (2008).
14. Ye M, Wysocki J, Gonzalez-Pacheco F R, Salem M, Evora K, Garcia-Halpin L, Poglitsch M, Schuster M, Batlle D. Hypertension. 60, 730-40 (2012).
15. Maier C, Schadock I, Haber P K, Wysocki J, Ye M, Kanwar Y, Flask C A, Yu X, Hoit B D, Adams G N, Schmaier A H, Bader M, Batlle D. J Mol Med (Berl). 95, 473-486(2017)
16. Jamil K, Pappas S C, Devarakonda K R. J Exp Pharmacol. December 20, 1-7 (2017).

### Example 2: Additional Exemplary Embodiments

Synthesis:

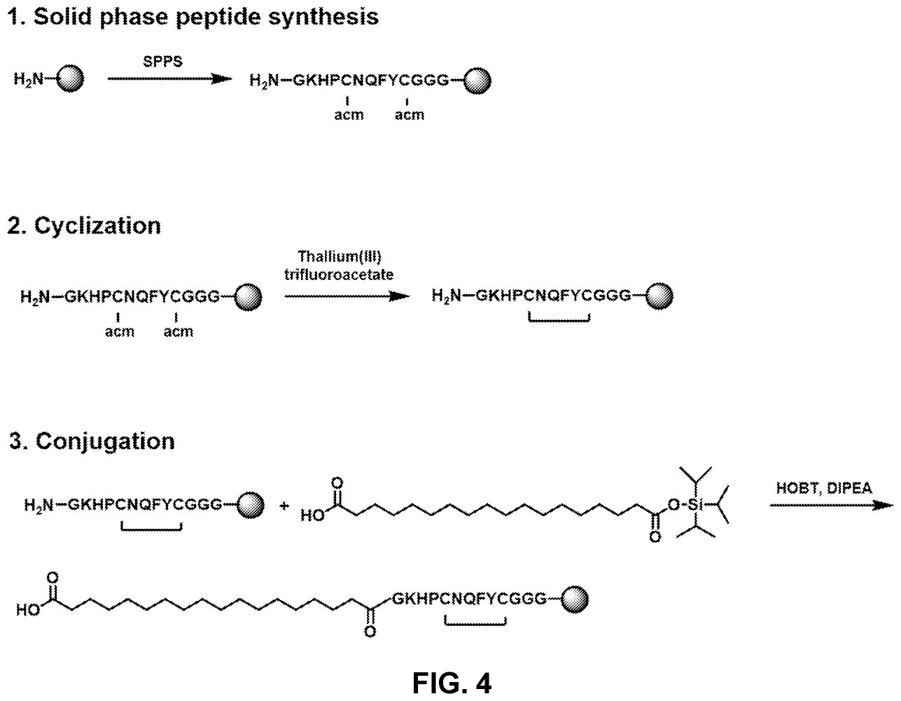

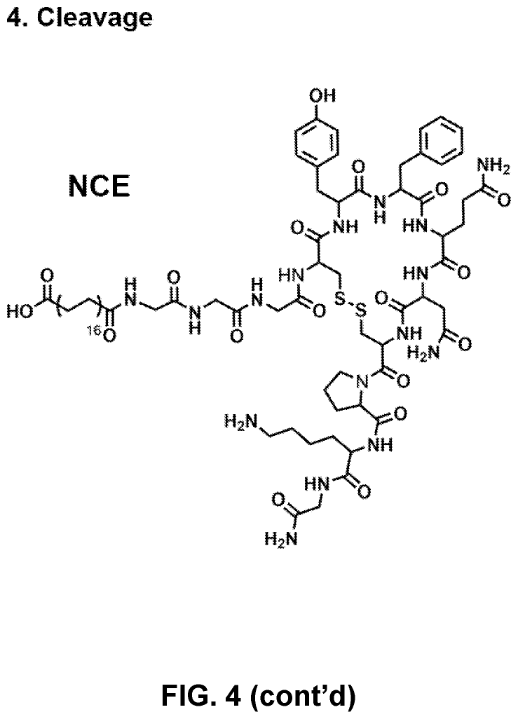

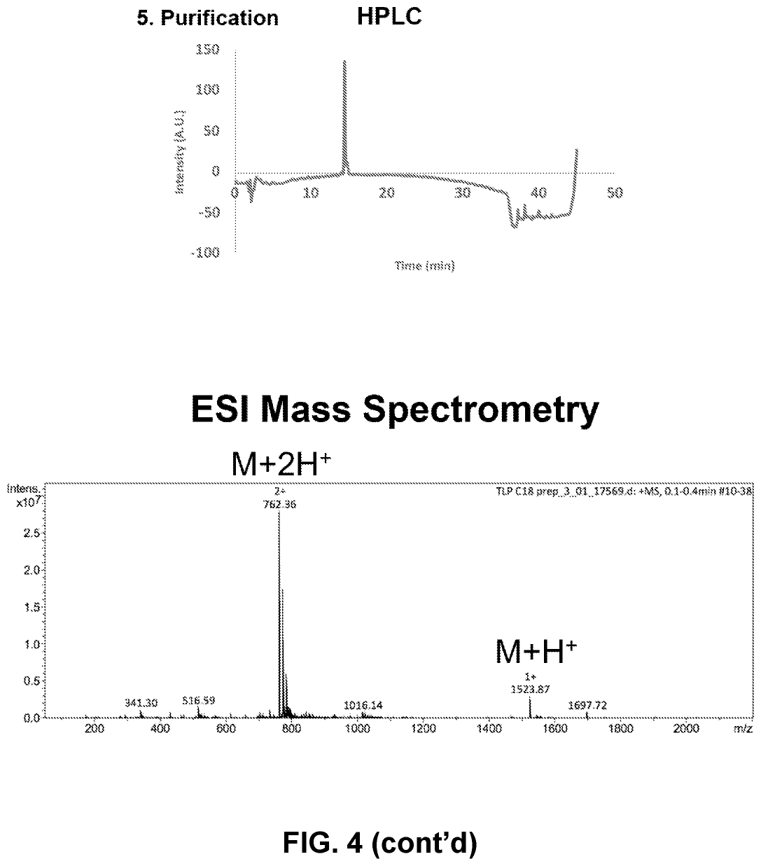

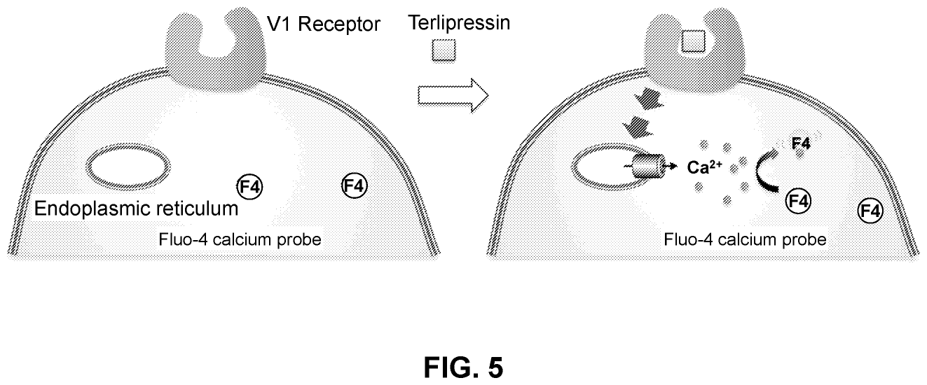

Peptide Synthesis and Conjugation (FIG. 4, Steps 1-3):

Terlipressin was synthesized using standard Solid Phase Peptide Synthesis (SPPS) procedures on an AAPPTec Focus XC automated synthesizer. The peptide was prepared on Rink amide MBHA resin (AAPPTec). A typical SPPS procedure involved Fmoc deprotection with 20% methylpiperidine in DMF (one 5 min deprotection followed by one 15 min deprotection), and 45 min amide couplings using 3.75 eq of the Fmoc-protected, and side chain-protected amino acid, 4 eq of HBTU and 8 eq of DIPEA.

Removal of Acm protecting group from the cysteine residues and formation of the disulfide bond was done on resin by stirring it with a thallium trifluoroacetate 1.2 eq in DMF (twice for 40 min).

The peptide was conjugated to 1,18-octadecanedioic acid (Elevance Renewable Sciences, Inc.) mono-protected with triisopropylsilyl ether (TIPS) manually with the same conditions used above.

Following completion of the synthesis, peptides were cleaved from the resin by treatment with TFA/H₂O/TIPS in a 9.5:2.5:2.5 ratio for 2 h. The conjugates were then precipitated in cold ether and purified by RP-HPLC.

Chemical Characterization (FIG. 4, Steps 4-5)

The identity of the conjugate was confirmed by ESI-MS and purities were verified by observation of a single peak in analytical RP-HPLC chromatograms.

In Vitro Data:

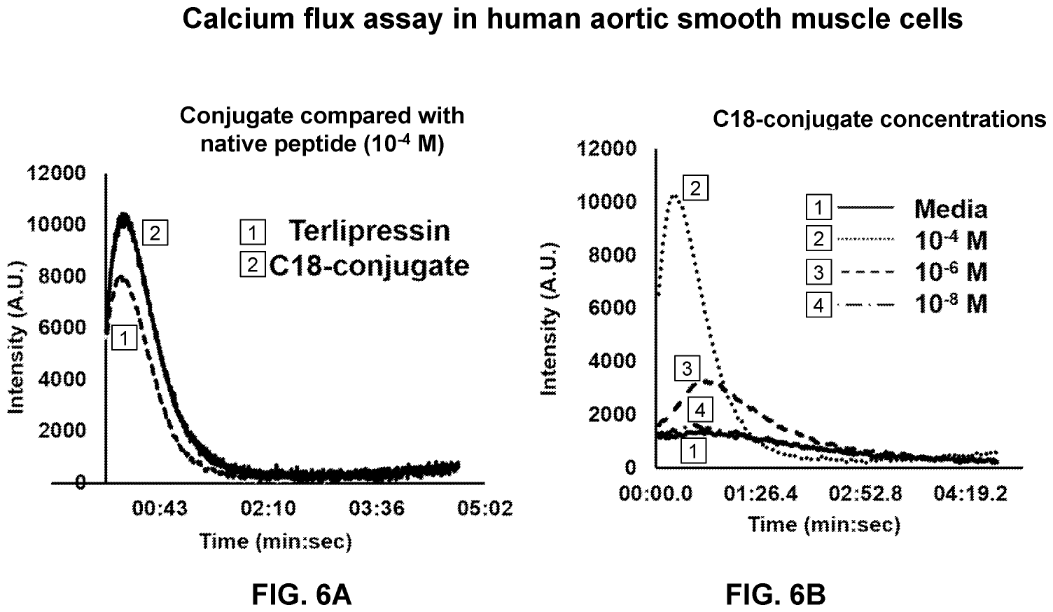

Cell Culture Assays (FIGS. 6A-6B and 1C):

Primary human aortic smooth muscle cells (ATCC, PCS-100-012 cell line) were cultured in vascular cell basal medium supplemented with rhFGF-basic 5 ng/mL, rh insulin 5 μg/mL, ascorbic acid 50 μg/mL, L-glutamine 10 mM, rh EGF 5 ng/mL, fetal bovine serum 5%.

Calcium influx assays were performed using Fluo-4 direct calcium assay kit (ThermoFisher) on cells between passage numbers 3 to 6. To this end, cells were passaged and plated in a 96-well black tissue culture plate in a concentration of 10,000 cells per well followed by incubation in 37° C. and 5% CO2 for 48 hours. Cells were loaded with the dye and incubated for 1 hr at 37° C. and 5% CO2. Then, terlipressin peptide or terlipressin-ODDA was added to the wells in concentrations ranging from 10⁻⁸ to 10⁻⁴ M, and fluorescence was measured immediately on a Perkin Elmer EnSpire 2300 plate reader. Signal was measured at 530 nm following excitation at 488 nm at 999 time points over a 5 min period.

The increase in signal following the addition of the triggers indicates that these materials can indeed bind to and activate the V1 receptor on the cell surface of SMCs.

Efficacy:

Blood Pressure Measurements (FIGS. 7A-7B):

The effects of C18-terlipressin were studied in C57BL/6 mice. The animals were anesthetized using ketamine for the duration of the measurements. Anesthetized animals were put into holders, placed on warming pads and attached to occlusion tail cuffs connected to CODA® high throughput noninvasive blood pressure system (Kent Scientific Corporation). Baseline systolic pressure was measured for 5-10 minutes following by injection of either terlipressin peptide, C18-terlipressin conjugate or PBS as negative control. Materials were in injected at a volume of up to 200 μl with concentrations ranging from 0.1 to 10 μg/gr body weight. Measurements were continued for another 20 minutes.

While the PBS did not have any significant effect on the measured blood pressure, both terlipressin and C18-terlipressin caused a clear increase in pressure for the duration of the measurements.

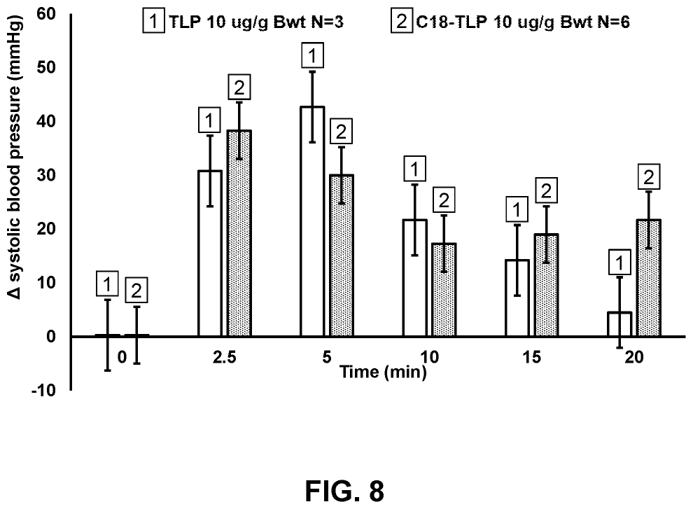

Blood Pressure Plots (FIG. 8):

The increase in systolic blood pressure at different time points following injections of terlipressin or C18-terlipreesin, as compared to the respective baselines, were compared.

It is evident that animals injected with terlipressin show a peak in the measured blood pressure 5 minutes following the injection, and continuous drop in pressure for the rest of the experiment, while animals injected with the C18 conjugate display more stable pressure over time.

Pharmacokinetics:

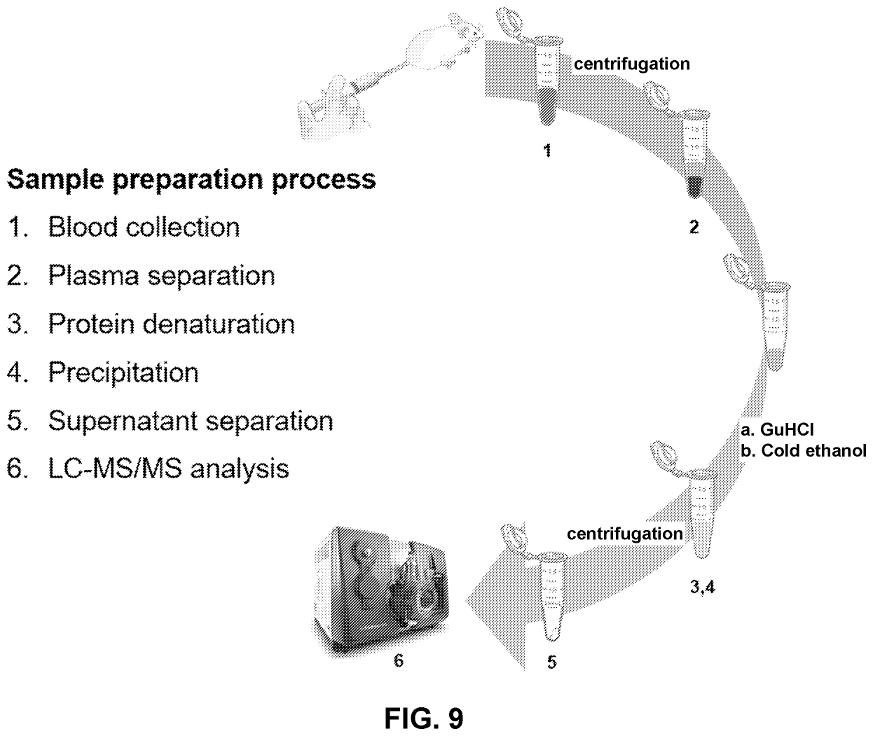

Blood Sample Processing (FIG. 9):

Blood samples were drawn from the animals (approximately 100 μl) at different time points (1 hr, 3 hr, 6 hr, 24 hr) following the injection. The plasma was separated from the whole blood by centrifugation at 7,000 RPM for 10 minutes and the supernatant was transferred to a new tube. To separate the peptides or peptide conjugates from the plasma proteins, the proteins were denatured using a 7M guanidinium hydrochloride solution mixed 1:1 with the plasma and incubated at room temperature for 15 minutes. Subsequently, to the solution was diluted 10-fold with cold ethanol and the samples were incubated at −20° C. After 1 hour incubation the samples were centrifuged at 10,000 RPM for 10 minutes to precipitate the proteins, and the supernatant was transferred to a new tube. Samples were kept at −80° C. until analyzed.

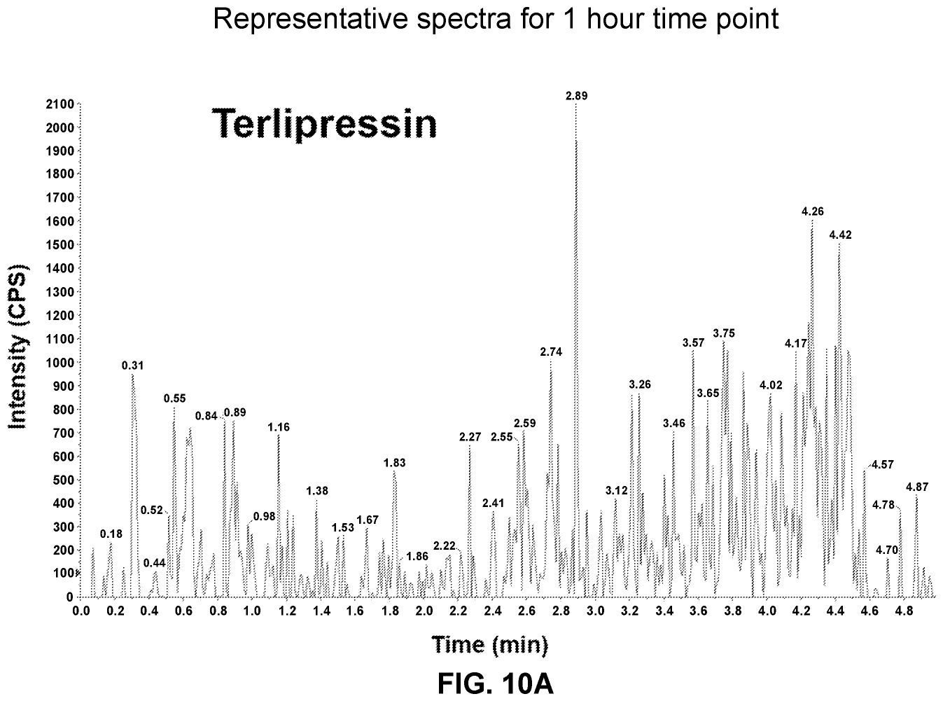

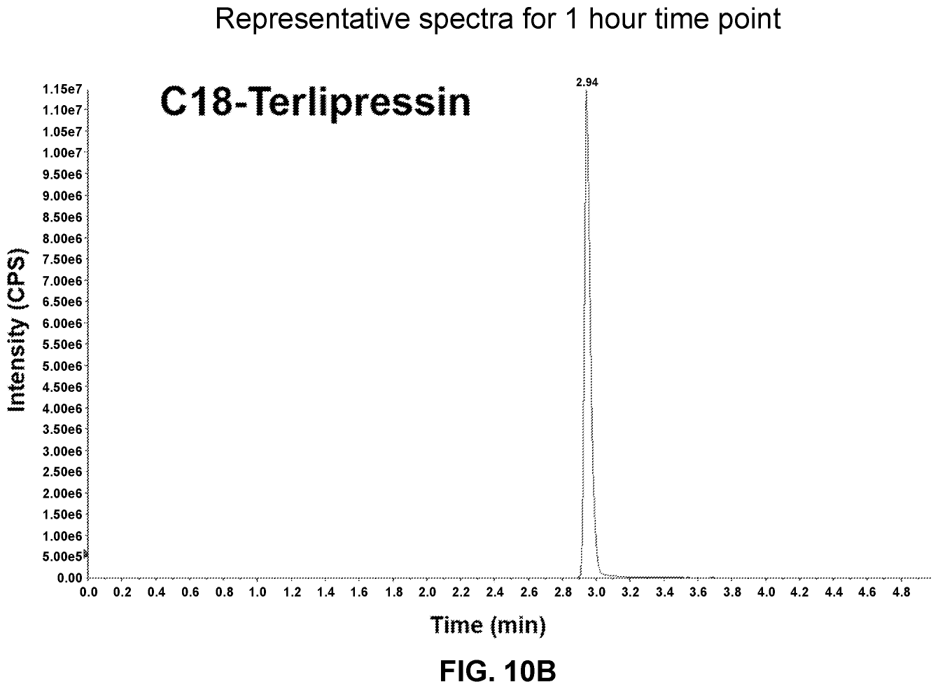

Liquid Chromatography with Tandem Mass Spectrometry (LC-MS-MS) (FIGS. 10A-10B):

5-20 μl of the ethanol solution were injected to a QTRAP 6500+LC-MS/MS system (SCIEX) for detecting the drug concentration. Mass spectra were obtained and the areas under the curves were evaluated against a calibration curve made formerly. Even at the earliest examined time point of 1 hour, no terlipressin was detected at all and only background-level noise can be seen. On the other hand, C18-terlipressin gave very sharp and clear peaks, even at the 24 hour time point.

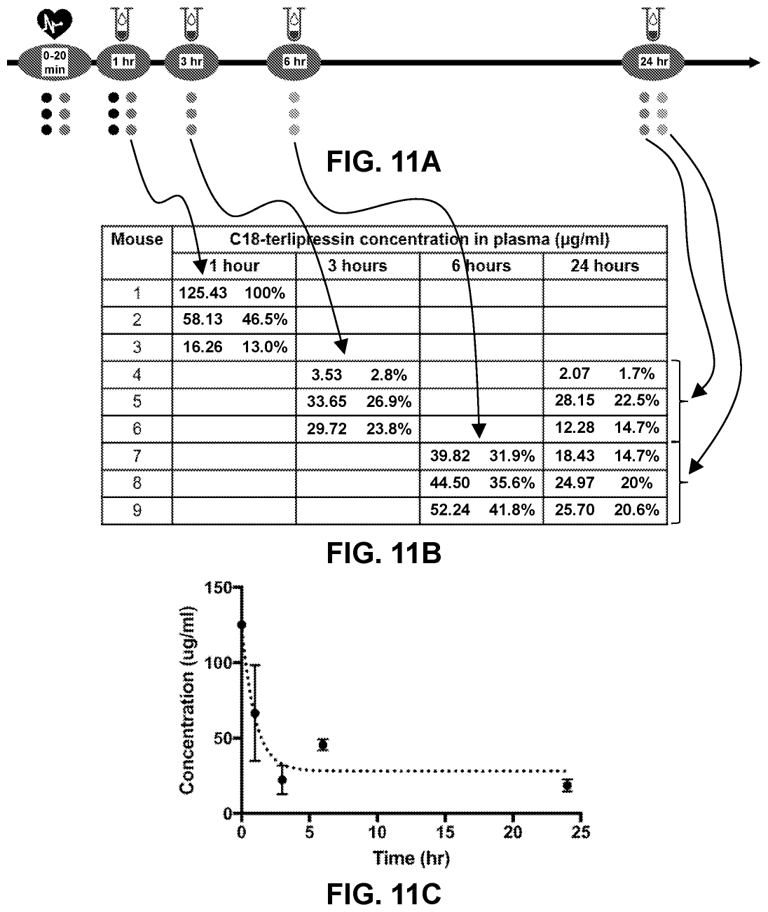

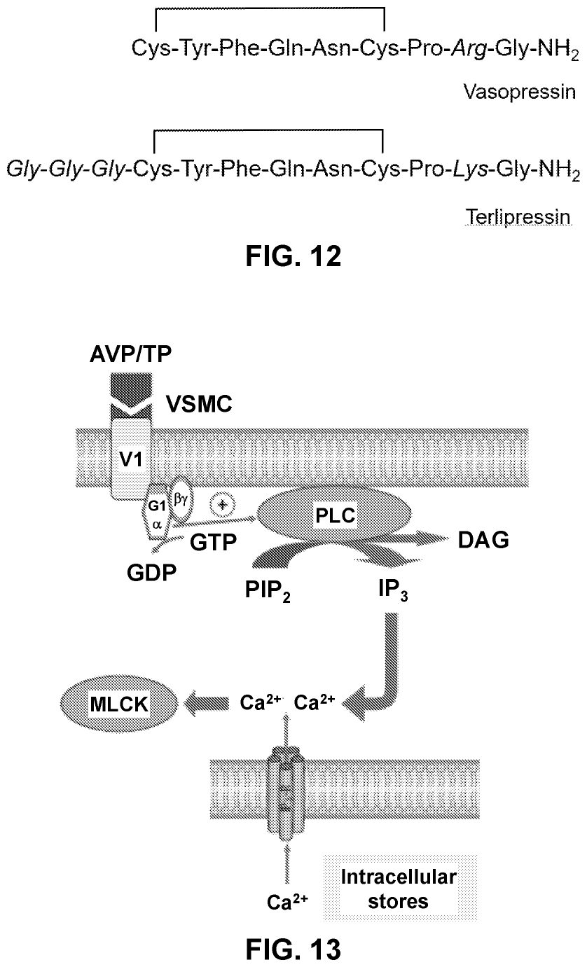

Lifetime (FIGS. 11A-11C):

The illustration depicts the experiment timeline. Blood pressure measurements were taken for the first 20 minutes following drug injection, and subsequently blood samples were drawn at 1, 3, 6 and 24 hours. The different colors represent different groups of animals that were used.

The plasma concentrations as calculated by the method described above are presented in the table, next to the percentage of remaining drug compared to the moment of injection (125 μg/ml). The results were plotted as a lifetime curve. The C18-terlipressin half-lifetime is accordingly around 1 hour, compared to the known half-life time of the peptide itself which is mere minutes.

Additional Description:

Hepatorenal syndrome (HRS) is the most frequent life-threatening complication of advanced liver failure and cirrhosis with critically poor prognosis and no approved drug in the U.S. The platform technology corresponds to payload armed lipid metabolites, or PALM. The composition of the treatment composition, according to embodiments, corresponds to octadecanedioic acid (ODDA) conjugates. The treatment strategy involves engaging fatty acid transport proteins via naturally evolved interactions. A useful compound is ODDA conjugated with the vasoconstrictor peptide drug terlipressin. Terlipressin suffers from poor PK in current formulations.

Data demonstrations that a terlipressin conjugate, according to certain embodiments of compounds and compositions disclosed herein, can stimulate response in cultured smooth muscle cells. Rise in blood pressure was observed in mice following IP injections. A method for PK investigation is developed. Additional studies include long-term blood pressure measurements using radio-telemetry. Antidiuretic effects of the drug in animal model are studied as well. Benefits of the compositions disclosed herein include a new therapy to benefit patients, allowing an immediate and easily delivered treatment without the need of a specialized physician, sophisticated equipment or surgery.

Scientific rationale: Stimulation of V1 receptors in vascular smooth muscle cells (SMCs) by terlipressin mediates actin-myosin interactions by increasing intracellular calcium concentrations through various mechanisms including activation of receptor-operated calcium channels, voltage-gated calcium channels via protein kinase C (PKC), and emptying of intracellular calcium stores.

SMCs are the cellular components of the normal blood vessel wall that provide structural integrity and regulate the diameter by contracting and relaxing dynamically in response to stimuli.

Terlipressin is approved in Europe and other countries, but not in the US. It is currently in Phase 3 trials in the US. It has validated targets. Typical of a small molecule peptide, it suffers from rapid clearance, poor PK.

ODDA-Terlipressin, a compound according to certain embodiments of compounds and compositions disclosed herein, provides extended half-life.

The market for Terlipressin (HRS indication) is estimated at >$300M/yr¹. There are 3.9M chronic liver disease patients in the U.S. Approx.

Other complications and diseases that may be treated by embodiments of compounds and compositions disclosed herein include: bleeding esophageal varices; septic shock hyponatremia; management of low blood pressure; and central diabetes insipidus.

Terlipressin is a medicine similar to a naturally occurring hormone present in the body, known as antidiuretic hormone (ADH) or vasopressin. ADH has two main effects in the body. Firstly, it causes narrowing of blood vessels (vasoconstriction), thereby limiting blood flow to a particular area of the body. It also acts on receptors in the kidney to retain water in the body, which helps to prevent excessive loss of water in the urine. Conventionally, administration of terlipressin is via IV and is well characterized in terms of ADME, and it is currently in Phase 3 trials. The half life of terlipressin following IV administration is less than 6 minutes.

Side-effects of the parent peptide terlipressin include: caution in people with hypertension; headache; bradycardia; hypertension; diarrhea; abdominal cramps. Side-effects of C18 diacid are: known human metabolite.

The effect of terlipressin-ODDA on blood pressure, compared to native terlipressin, was evaluated in a small pilot study in mice. We employed a tail-cuff system coupled with a volume pressure recording sensor to allow accurate non-invasive blood pressure measurements in mice. Terlipressin conjugates were administrated to C57BL mice by intraperitoneal injection (IP) at the Batlle lab. The ODDA-terlipressin conjugates were shown to increase the systolic blood pressure of the mice, compared with negative control (vehicle) and positive control (terlipressin peptide) Market need: HRS is the leading cause of hospitalizations among all patients with chronic liver disease. Therefore, management of patients with HRS is time and resource intensive, representing significant costs to hospitals. In 2015, the total incidence of CLD in the US was 1.5% (3.9 million US adults), representing severe and consequential morbidity and mortality.

### Example 3: Terlipressin Brush Polymers for Vasoconstrictive Therapy

Terlipressin is an analogue of the naturally occurring peptide vasopressin that causes narrowing of blood vessels (vasoconstriction). It is a registered drug in Europe, Australia and parts of Asia, prescribed for patients with bleeding esophageal varices (bleeding from dilated veins in the food pipe leading to the stomach).

Bleeding esophageal varices: If the vessels in the liver are blocked due to liver damage, blood cannot flow properly through the liver. As a result, high pressure in the portal system develops. This increased pressure in the portal vein may lead to the development of large, swollen veins (varices) within the esophagus, stomach, rectum, or umbilical area. Varices can rupture and bleed, resulting in potentially life-threatening complications.

Bleeding of oesophageal varices is one of the most dramatic complications in gastroenterology and has a 20-50% mortality rate, closely related to failure to control initial bleeding or early rebleeding occurring in up to 30-40% of patients.

The only approved drugs to arrest variceal bleeding are vasopressin and terlipressin. Treatment with terlipressin is preferable due to better efficacy, longer effects and less adverse effects compared to vasopressin. Yet, treatment with terlipressin has several drawbacks.—The distribution half-life is 8 minutes, while the elimination half-life is 6 minutes. Consequently, terlipressin has to be administered by intermittent intravenous dosing schedule of approximately every 4-6 hours in doses of 1-2 mg per injection, until bleeding is under control. Duration of treatment can last up to 3 days.

Vascular smooth muscle cells (VSMCs) are the cellular components of the normal blood vessel wall that provide structural integrity and regulate the diameter by contracting and relaxing dynamically in response to vasoactive stimuli. V1 receptors are present on VSMCs. Upon activation of these receptors by vasopressin analogs, a G-protein-mediated release of calcium from intracellular stores within the sarcoplasmic reticulum occurs that subsequently activates membrane channels, allowing for the influx of extracellular calcium to further regulate calcium balance. Calcium concentration is the primary determinate of actin-myosin cross-bridge cycling and force of contraction in vascular smooth muscle; thus, increases in calcium concentration lead to systemic vasoconstriction.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

The term “and/or” is used herein, in the description and in the claims, to refer to a single element alone or any combination of elements from the list in which the term and/or appears. In other words, a listing of two or more elements having the term “and/or” is intended to cover embodiments having any of the individual elements alone or having any combination of the listed elements. For example, the phrase “element A and/or element B” is intended to cover embodiments having element A alone, having element B alone, or having both elements A and B taken together. For example, the phrase “element A, element B, and/or element C” is intended to cover embodiments having element A alone, having element B alone, having element C alone, having elements A and B taken together, having elements A and C taken together, having elements B and C taken together, or having elements A, B, and C taken together.

The term “t” refers to an inclusive range of values, such that “X±Y,” wherein each of X and Y is independently a number, refers to an inclusive range of values selected from the range of X−Y to X+Y.

Every compound, formulation, composition, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A compound characterized by formula (FX1):
A¹-X¹—X²-A² (FX1);
wherein:
A¹ is a carboxylic acid group, a carboxylate anion, or a carboxylate ester;
X¹ is a substituted or unsubstituted and saturated or unsaturated C₁-C₅₀ aliphatic group;
X² is a linker group selected from the group consisting of a direct bond, an organic group, —O—, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and
A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin, or
a pharmaceutically acceptable salt thereof.

2. The compound of claim 1 or a pharmaceutically acceptable salt thereof, wherein X² is selected from the group consisting of an amide group, an ester group, a disulfide group, a carbamate group, a carbonate group, a ketone group, and a combination thereof.

3-9. (canceled)

10. The compound of claim 1 or a pharmaceutically acceptable salt thereof, wherein X¹ is (CH₂)₁₂, (CH₂)₁₄, (CH₂)₁₆, (CH₂)₁₈, (CH₂)₂₀, or (CH₂)₂₂.

11. (canceled)

12. The compound of claim 1 or a pharmaceutically acceptable salt thereof, wherein A² is terlipressin, vasopressin, omipressin, desmopressin, lypressin, or felypressin

13. (canceled)

14. The compound of claim 1 or a pharmaceutically acceptable salt thereof, wherein the peptide A² comprises a sequence having 80% or greater sequence homology of Seq. ID. 1 (GGGCYFQNCPKG).

15. The compound of claim 14 or a pharmaceutically acceptable salt thereof, wherein the peptide A² comprises the amino acid sequence of Seq. ID. 1 (GGGCYFQNCPKG).

16. (canceled)

17. (canceled)

18. The compound of claim 1 or a pharmaceutically acceptable salt thereof, characterized by the formula (FX2):

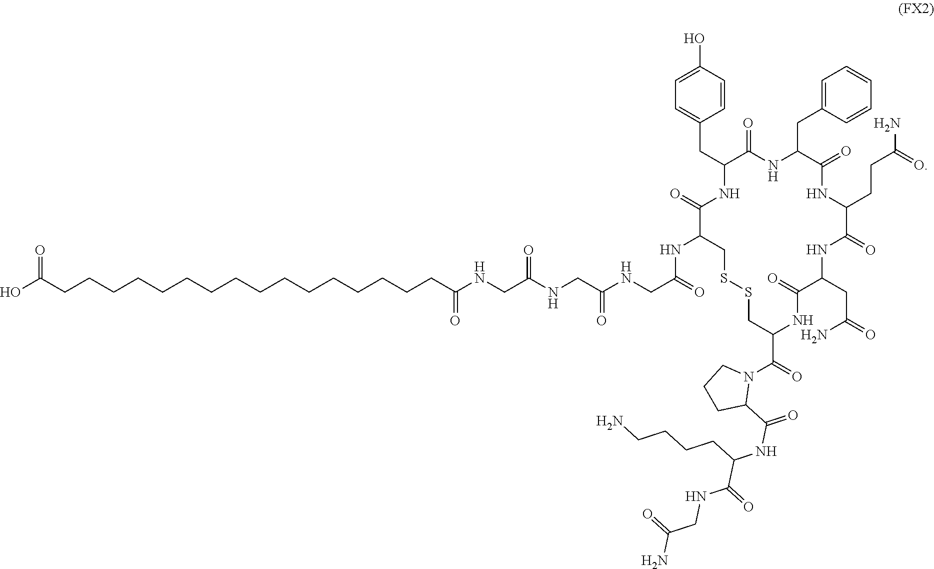 [SMILES: NCCCCC(NC(=O)C1CCCN1C(=O)C1CSSCC(NC(=O)CNC(=O)CNC(=O)CNC(=O)CCCCCCCCCCCCCCCCC(=O)O)C(=O)NC(Cc2ccc(O)cc2)C(=O)NC(Cc2ccccc2)C(=O)NC(CCC(N)=O)C(=O)NC(CC(N)=O)C(=O)N1)C(=O)NCC(N)=O]

19. (canceled)

20. A pharmaceutical composition comprising the compound of claim 1 or a pharmaceutically acceptable salt thereof.

21. The pharmaceutical composition of claim 20 further comprising a protein, wherein the protein is human serum albumin or a protein whose sequence is at least 50% equivalent to that of human serum albumin.

22-32. (canceled)

33. A method of treating or managing a condition in a living subject, the method comprising steps of: administering to the subject a pharmaceutical composition; wherein the pharmaceutical composition comprises:
a compound characterized by formula (FX1):
A¹-X¹—X²-A² (FX1);
wherein:
A¹ is a carboxylic acid group, a carboxylate anion, or a carboxylate ester;
X¹ is a substituted or unsubstituted and saturated or unsaturated C₁-C₅₀ aliphatic group;
X² is a linker group selected from the group consisting of a direct bond, an organic group, —O—, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and
A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin.

34. The method of claim 33, wherein the condition is selected from the group consisting of hepatorenal syndrome, low blood pressure, bleeding esophageal varices, septic shock paracentesis-induced circulatory dysfunction, a condition or disease that can be treated using vasoconstriction or albumin-mediated vasoconstriction, and any combination thereof.

35. (canceled)

36. (canceled)

37. The method of claim 33, wherein the step of administering comprises intravenous administration in the living subject.

38. The method of claim 33, wherein the step of administering is performed at a frequency of greater than 6 hours.

39. (canceled)

40. The method of claim 33, wherein the pharmaceutical composition has a half-life that is greater than 1 hour in the blood of the subject after being administered.

41. The method of claim 33, wherein the pharmaceutical composition causes an increased systolic blood pressure in the subject for at least 30 minutes after being administered.

42. (canceled)

43. (canceled)

44. A method for making the compound of claim 1, the method comprising:
conjugating a molecule comprising A¹-X¹—X²— with A², thereby forming the compound;
wherein the compound is characterized by formula (FX1):
A¹-X¹—X²-A² (FX1);
wherein:
A¹ is a carboxylic acid group, a carboxylate anion, or a carboxylate ester;
X¹ is a substituted or unsubstituted and saturated or unsaturated C₁-C₅₀ aliphatic group;
X² is a linker group selected from the group consisting of a direct bond, an organic group, —O—, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and
A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin.

45. The method of claim 44, wherein the molecule is conjugated to amine group of A².

46-49. (canceled)

50. A compound characterized by formula (FX10):
(A¹-X¹-A²-)ₙA² (FX10);
wherein:
each A¹ is independently a carboxylic acid group, a carboxylate anion, or a carboxylate ester;
each X¹ is independently a substituted or unsubstituted and saturated or unsaturated C₁-C₅₀ aliphatic group;
each X² is independently a linker group selected from the group consisting of a direct bond, an organic group, —O—, —S—, —S(═O)—, —S(═O)₂—, —S—S—, —N═, ═N—, —N(H)—, —N═N—N(H)—, —N(H)—N═N—, —N(OH)—, —N(═O)—, and any combination thereof; and
A² is a peptide, the peptide being terlipressin or a substituted or unsubstituted derivative, a substituted or unsubstituted natural or synthetic analogue, a substituted or unsubstituted variant, a substituted or unsubstituted isomer, or a substituted or unsubstituted fragment of terlipressin; and
n is an integer selected from the range of 1 to 10,
or a pharmaceutically acceptable salt thereof.

51. The compound of claim 50 or a pharmaceutically acceptable salt thereof, wherein n is greater than 1 and each (A¹-X¹—X²—) is covalently bound to a unique binding site of A².

52. (canceled)

53. A pharmaceutical composition comprising the compound of claim 50 or a pharmaceutically acceptable salt thereof.

54. (canceled)

55. (canceled)

56. The pharmaceutical composition of claim 53, further comprising a protein, wherein the protein is human serum albumin or a protein whose sequence is at least 50% equivalent to that of human serum albumin.
